(19) United States
(12) Patent Application Publication
(10) Pub. No.: US 2024/0115327 A1
(43) Pub. Date: Apr. 11, 2024

(54) AIMING SYSTEM AND METHOD OF USE THEREOF

(71) Applicant: SAVFE Co., Ltd., Taipei city (TW)

(72) Inventors: Chih Wei CHEN, Taipei City (TW); Hao Kai CHOU, New Taipei City (TW); Chih Min YANG, New Taipei City (TW)

(21) Appl. No.: 18/377,535

(22) Filed: Oct. 6, 2023

Related U.S. Application Data
(60) Provisional application No. 63/414,519, filed on Oct. 9, 2022.

(51) Int. Cl. A61B 34/20; A61B 90/00

(52) U.S. Cl. CPC A61B 34/20; A61B 90/37; A61B 2034/2055; A61B 2090/365

(57) ABSTRACT

An aiming system includes a positioner, an aiming device, and a processor. The positioner has a function of acquiring spatial information, and the aiming device is connected to the positioner, and the processor is connected to the positioner or the aiming device. A method of using the aiming system is also provided. Surgical guidance is more intuitive by directly combining the positioner with the aiming device.

### CROSS-REFERENCE TO RELATED APPLICATION

This application claims priority to U.S. Provisional Application Ser. No. 63/414,519, filed on Oct. 9, 2022, which is herein incorporated by reference in its entirety.

### BACKGROUND

### Field of Invention

The present disclosure relates to an aiming system and a method of use thereof, and in particular, to a surgical aiming system and a method of use thereof.

### Description of Related Art

Most of traditional infrared optical navigation systems need to fix a fiducial marker on a patient in an invasive manner. However, during the operation, positioning failure may occur due to collision displacement or contamination of the fiducial marker.

In addition, a traditional navigation tool often has to be identified by a tracker during using, which leads to inconvenient operation. Specifically, in order for the traditional navigation tool to be navigable, it needs to design a variety of dedicated trackable markers. As such, it is impossible to effectively integrate tracking markers with various tools and instruments in an operating room, resulting in poor compatibility.

Therefore, the existing technology needs to be improved.

### SUMMARY

One embodiment of the present disclosure provides an aiming system includes a positioner, an aiming device, and a processor. The positioner has a function of acquiring spatial information, and the aiming device is connected to the positioner, and the processor is connected to the positioner or the aiming device.

In some embodiments, the aiming device includes an aimer, which includes at least one hollow channel disposed at a surface of the positioner.

In some embodiments, the positioner includes at least one sensing device disposed at one side of the positioner.

In some embodiments, an axial direction of the at least one hollow channel and a sight direction of the at least one sensing device are substantially parallel to each other.

In some embodiments, the aiming system further includes a prompt signal disposed at one end of the at least one hollow channel, and the prompt signal and the at least one sensing device are respectively located at opposite ends of the positioner.

In some embodiments, the prompt signal is annular and has a plurality of light sources, and the prompt signal is arranged around one end of the at least one hollow channel; when a portion of the light sources of the prompt signal emits light, the aiming device moves toward a location where the portion of the light sources are located until the light disappears to complete alignment.

In some embodiments, the aiming system further includes a display connected to the aiming device, in which the display displays image including positioning prompt information, medical imaging information, a surgical plan, or a combination thereof.

In some embodiments, the display is disposed on the positioner, and the aimer is penetrated through the display.

In some embodiments, the aiming system further includes a prompt signal disposed at one end of the at least one hollow channel, and the prompt signal and the at least one sensing device are respectively located at opposite ends of the positioner.

In some embodiments, the display integrates the aiming device to display a medical image of a body surface range and/or its surroundings corresponding to the aimer at that time, and the medical image includes a visible light image of the body surface range and/or its surroundings, a fluoroscopic medical image of the body surface range and/or its surroundings, or a combination thereof.

In some embodiments, the display is disposed on the positioner, and the aimer is disposed on the display.

In some embodiments, the aiming device includes: a projection light source disposed at a surface of the positioner; a virtual aimer projected and displayed by the projection light source; a virtual prompt signal projected and displayed by the projection light source, and the virtual prompt signal presented at one end of the virtual aimer; and a virtual surgical information projected and displayed by the projection light source, and the virtual surgical information presented at one side of the virtual aimer.

In some embodiments, the aiming system further includes at least one handle disposed at one side of the positioner or the aiming device.

In some embodiments, the aiming system further includes a connecting rod disposed beneath the positioner or the aiming device, so that the aiming system is locked and fixed in space with any posture.

In some embodiments, the aiming system further includes a visible light source disposed at one side of the positioner or one side of the aiming device.

In some embodiments, the aiming device includes an aimer, which includes a hollow channel; the aiming system further includes a plurality of slide grooves arranged around outside the hollow channel in sequence; and the positioner includes a plurality of sensing devices respectively movably disposed in the plurality of slide grooves and movably arranged around the aimer.

In some embodiments, the aiming device further includes: a base, the plurality of slide grooves are arranged around an outer wall of the base in sequence; and a fine-tuning mechanism disposed between an outer wall of the hollow channel and an inner wall of the base, so that the hollow channel is movably penetrated through the base.

In some embodiments, the aiming device includes an aimer, which includes: a base; and a hollow channel movably penetrated through the base; and the positioner includes a plurality of sensing devices respectively disposed at an outer surface of the base.

In some embodiments, the base includes a ball joint, and the hollow channel is rotatably movably penetrated through the ball joint.

In some embodiments, the base includes a slide rail, and an axial direction of the hollow channel is parallel to an axial direction of the slide rail, and the hollow channel is movably penetrated through the base along the axial direction of the slide rail.

In some embodiments, the aiming system further includes a display, which includes a first side and a second side opposite to the first side; the aiming device includes an aimer, which includes: a hollow channel disposed on the display or penetrated through the display, and the hollow channel has a first end and a second end opposite to the first end, and the second end of the hollow channel is coplanar with a second end of the display; and the positioner is disposed on the display.

In some embodiments, the aimer further includes: a ball joint disposed at the first end of the hollow channel; and a suction cup disposed at one side of the ball joint opposite to the first end of the hollow channel.

In some embodiments, the aimer further includes a sensor disposed at the first end of the hollow channel.

In some embodiments, the positioner protrudes from a surface of the display by a first height, and the hollow channel protrudes from the surface of the display by a second height, and the first height is less than the second height.

In some embodiments, the positioner includes two sensing devices respectively disposed at both ends of the first side of the display, and there is a distance between the two sensing devices, and the hollow channel has a diameter less than or equal to the distance.

In some embodiments, the positioner is disposed on the first side of the display; and the aiming system further includes an observer disposed on the second side of the display to observe user behavior.

In some embodiments, the aiming system further includes a calibration group, which includes: a first calibration device, a second calibration device or a third calibration device. The first calibration device includes: a base having a first end and a second end opposite to the first end; a combination base disposed at the first end of the base, and one end of the display is detachably combined with the combination base; and a bump protruding from the second end of the base, and the bump having a calibration feature located in front of the first end of the hollow channel, in which the positioner is movably disposed on the first side of the display to perform calibration according to a position of the calibration feature. The second calibration device includes: a rod body having a first end and a second end opposite to the first end, the second end detachably penetrated through and in the hollow channel or sleeved outside the hollow channel; and a calibration feature disposed at the first end of the rod body, the calibration feature located in front of the first end of the hollow channel, in which the hollow channel moves or rotates along an axial direction to perform calibration according to a position of the calibration feature. The third calibration device, includes: a base having a first end and a second end opposite to the first end; a combination base disposed at the first end of the base, one end of the display detachably combined with the combination base; a rod body having a first end and a second end opposite to the first end, the second end of the rod body sliding or rotating along an axial direction of the base; and a calibration feature disposed at the first end of the rod body, the calibration feature located in front of the first end of the hollow channel, in which the positioner is fixed, and the calibration feature on the rod body can slide and rotate to perform calibration.

In some embodiments, the positioner includes a first side, a second side opposite to the first side, and at least one camera disposed at the first side; the aiming device includes an aimer, which includes: a hollow channel disposed on the positioner, and the hollow channel has a first end and a second end opposite to the first end, and the second end is coplanar with a second side of the positioner, and the first end of the hollow channel protrudes from the first side of the positioner; and a calibration feature disposed at the first end of the hollow channel, and a distance between the calibration feature and the first side of the positioner is greater than or equal to a shortest focal length of the at least one camera.

In some embodiments, the aiming device includes an aimer, which includes a hollow channel disposed at one side of the positioner, in which an inner diameter of the hollow channel is greater than or equal to an outer diameter of an instrument, in which the aimer further includes: an inflatable structure disposed at an inner wall of the hollow channel; at least one vacuum feature disposed at the inner wall of the hollow channel, and when the at least one vacuum feature is in a vacuum state, the at least one vacuum feature presents a hardened state, and when the at least one vacuum feature is in a non-vacuum state, the at least one vacuum feature presents a soft state; an adapter, the adapter being annular and disposed at the inner wall of the hollow channel; an inner diameter of the adapter being greater than or equal to the outer diameter of the instrument; the adapter and a dedicated drape, the adapter being annular and disposed at the inner wall of the hollow channel; the inner diameter of the adapter being greater than or equal to the outer diameter of the instrument; the dedicated drape being disposed between the hollow channel and the adapter; a fixed rod and at least one displacement detection roller, one end of the fixed rod being pivotally connected to the inner wall of the hollow channel, another end of the fixed rod being movably disposed in a cavity of the hollow channel; the at least one displacement detection roller being disposed at the inner wall of the hollow channel, and the at least one displacement detection roller being not located over or beneath the fixed rod; or a scanner disposed at the inner wall of the hollow channel, the scanner acquiring a depth of the instrument through the hollow channel by scanning and then calculating by the processor.

One embodiment of the present disclosure also provides a method of using an aiming system, which includes providing the aforementioned aiming system; scanning a medical image of an object and determining whether there is a surface artificial feature on the object, if so, scanning the surface artificial feature using the aiming system, and fitting the surface artificial feature on the medical image and the surface artificial feature scanned and acquired by the scanning system to acquire a coordinate conversion relationship; if not, scanning an object surface feature of the object using the aiming system, and fitting the object surface feature on the medical image and the object surface feature scanned and acquired by the scanning system to acquire the coordinate conversion relationship; and operating the aiming system together with an instrument.

In some embodiments, after the step of determining whether there is the surface artificial feature on the object, the method further includes: selecting a guidance prompt interface, which includes displaying through a prompt signal of the aiming system, displaying through a display of the aiming system, displaying through a projection light source projection of the aiming device of the aiming system, or a combination of one or more thereof.

In some embodiments, after the step of determining whether there is the surface artificial feature on the object, the method further includes: selecting a medical image display interface, which includes displaying a display of the aiming system, displaying a projection light source projection of the aiming device of the aiming system, or a combination of one or more thereof.

In some embodiments, the display displaying includes a virtual reality displaying or a mixed reality displaying, in which the virtual reality displaying is a digitally reconstructed radiograph updated by the aiming system through the coordinate conversion relationship and a spatial coordinate of the positioner of the aiming system at that time and displayed on the display, in which the mixed reality displaying is acquired by superimposing a color image acquired through the positioner of the aiming system on the digitally reconstructed radiograph projected by the virtual reality displaying, so that the digitally reconstructed radiograph and the color image are superimposed.

### BRIEF DESCRIPTION OF THE DRAWINGS

Aspects of the present disclosure are best understood when the following detailed description is read with the accompanying figures. It is noted that, in accordance with the standard practice in the industry, various features may not be drawn to scale. In fact, dimensions of the various features may be arbitrarily increased or reduced for clarity of discussion. In order to make the above and other objects, features, advantages and embodiments of the present disclosure more clear and understandable, the description of the accompanying drawings are as follows:

FIG. 1 is a schematic perspective view of a scanning side of an aiming system according to some embodiments of the present disclosure.

FIG. 2 is a schematic perspective view of an alignment side of an aiming system according to some embodiments of the present disclosure.

FIG. 3 is a schematic diagram of a sight and an axial space of an aiming system according to some embodiments of the present disclosure.

FIG. 4 is a schematic diagram of an aiming system equipped with a physical aimer and a hollow channel according to some embodiments of the present disclosure.

FIG. 5 is a schematic diagram of a scanning side of an aimer combined with a hollow display according to some embodiments of the present disclosure.

FIG. 6 is a schematic diagram of an alignment side of an aimer combined with a hollow display according to some embodiments of the present disclosure.

FIG. 7 is a schematic diagram of an image display panel on an aimer according to some embodiments of the present disclosure.

FIG. 8 is a schematic diagram of an aimer combined with a display according to some embodiments of the present disclosure.

FIG. 9 is a schematic diagram of a virtual aimer according to some embodiments of the present disclosure.

FIG. 10 is a schematic diagram of a use of an aiming system with a handle according to some embodiments of the present disclosure.

FIG. 11 is a schematic diagram of a use of an aiming system with a connecting rod according to some embodiments of the present disclosure.

FIG. 12 illustrates a schematic diagram of a use of an aiming system with a visible light source according to some embodiments of the present disclosure.

FIG. 13 is a schematic diagram of a non-coaxial arrangement in which a positioner of an aiming system is movable and an aimer thereof is immovable according to some embodiments of the present disclosure.

FIG. 14A is a schematic diagram of a non-coaxial arrangement in which a positioner of an aiming system is immovable and an aimer thereof is rotatable and movable according to some embodiments of the present disclosure.

FIG. 14B is a schematic diagram of a non-coaxial arrangement in which a positioner of an aiming system is immovable and an aimer thereof is forwardly and backwardly movable according to some embodiments of the present disclosure.

FIG. 15A is a schematic diagram of an aimer combined with a display and a positioner of an aiming system according to some embodiments of the present disclosure.

FIG. 15B is a schematic diagram of an aimer combined with a display, a positioner and a movable interface of an aiming system according to some embodiments of the present disclosure.

FIG. 15C is a schematic diagram of an aimer combined with a display, a positioner and a sensor of an aiming system according to some embodiments of the present disclosure.

FIGS. 16A and 16B are schematic diagrams illustrating operation of an aiming system with a fine-tuning mechanism according to some embodiments of the present disclosure.

FIG. 17 is a schematic diagram of dual positioners, an aimer and a display of an aiming system according to some embodiments of the present disclosure.

FIG. 18 is a schematic diagram of an aiming system with an observer according to some embodiments of the present disclosure.

FIG. 19 is a schematic diagram of an aiming system with a calibration feature on an aimer according to some embodiments of the present disclosure.

FIG. 20 is a schematic diagram of an aiming system with a calibration block according to some embodiments of the present disclosure.

FIG. 21 is a schematic diagram of an aiming system with a calibration rod according to some embodiments of the present disclosure.

FIG. 22 is a schematic diagram of an aiming system with a calibration block and a calibration rod according to some embodiments of the present disclosure.

FIG. 23 is a schematic diagram of a contoured design of an aimer and an instrument according to some embodiments of the present disclosure.

FIG. 24 is a schematic diagram of an aimer equipped with a vacuum feature according to some embodiments of the present disclosure.

FIG. 25 is a schematic diagram of an aimer equipped with a vacuum feature according to some embodiments of the present disclosure.

FIG. 26A is a schematic diagram of an aimer equipped with an adapter according to some embodiments of the present disclosure.

FIG. 26B is a schematic diagram of an aimer equipped with an adapter and a dedicated drape according to some embodiments of the present disclosure.

FIG. 27 is a schematic diagram of an aimer with a surgical instrument displacement detection function according to some embodiments of the present disclosure.

FIG. 28 is a schematic diagram of an aimer with a surgical instrument displacement detection function according to some embodiments of the present disclosure.

FIG. 29 is a schematic diagram of an aimer with a surgical instrument displacement detection function according to some embodiments of the present disclosure.

FIG. 30 illustrates a flow chart of a use of an aiming system according to some embodiments of the present disclosure.

### DETAILED DESCRIPTION

In order to make the description of the present disclosure more detailed and complete, an illustrative description is provided below for implementation aspects and specific embodiments of the present disclosure, but this is not the only form of implementing or using the specific embodiments of the present disclosure. The embodiments disclosed below can be combined or replaced with each other under beneficial circumstances, and other embodiments can be added to one embodiment without further description or explanation. In the following description, many specific details will be set forth in detail to enable the reader to fully understand the following embodiments. However, the embodiments of the present disclosure may be practiced without these specific details.

In addition, spatially relative terms, such as “beneath”, “on”, etc., are used to conveniently describe a relative relationship between one element or feature and another element or feature in the drawings. These spatially relative terms are intended to encompass different orientations of the device in use or operation in addition to the orientation depicted in the drawings. The device may be otherwise oriented (e.g., rotated 90 degrees or at other orientations), and spatially relative descriptions used herein interpreted accordingly.

In this description, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but do not exclude one or more of other features, regions, integers, steps, operations, elements, components, and/or groups thereof described or additionally.

The present disclosure is an aiming system, which includes a positioner, an aimer, a processor and a prompt signal. Following embodiments will illustrate interactive relationships between components as well as features of combined structures and usage processes. It should be understood that the above noun is one of terms of commonly used definitions. The aimer may also be called a synonym for an aiming dilator, an alignment dilator, a dilator, a sleeve pipe, a guide dilator, etc.

A function of the positioner is to acquire current spatial information and surface information of a patient during surgery and to transmit it to the processor. The current spatial information includes but is not limited to one or a combination of an image of a range viewed by the positioner, spatial coordinates of the positioner, and coordinates of an object in the range viewed by the positioner. The surface information includes but is not limited to one or a combination of physiological information on the patient's body surface and an artificial feature fixed on the patient's body surface.

After receiving the surface information, the processor can register the surface information acquired by the positioner with a patient's medical image, and calculate a coordinate relationship between the positioner and the patient's medical image, and then generate a prompt signal, which can be used to guide and align the aimer to a surgical target range.

Several embodiments and experimental examples are provided below to further elaborate on the aiming system of the present disclosure. However, those are only for illustrative purposes and are not used to limit the present disclosure. The scope of protection of the present disclosure shall be subject to the scope of appended claims.

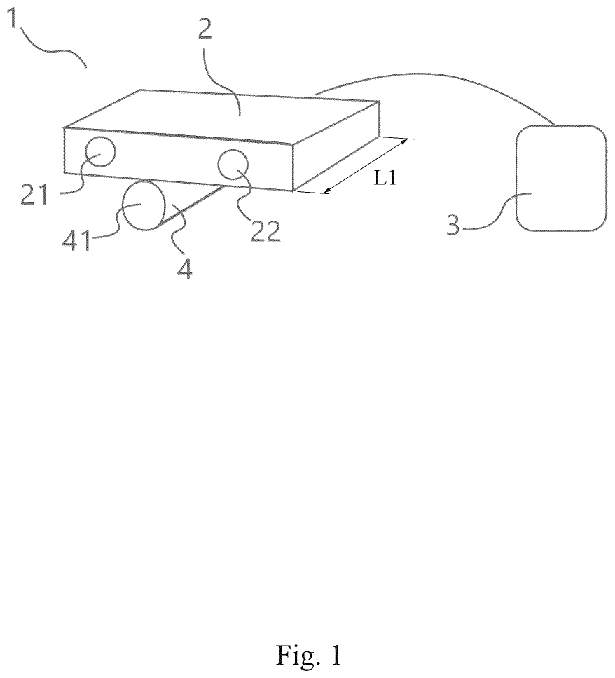

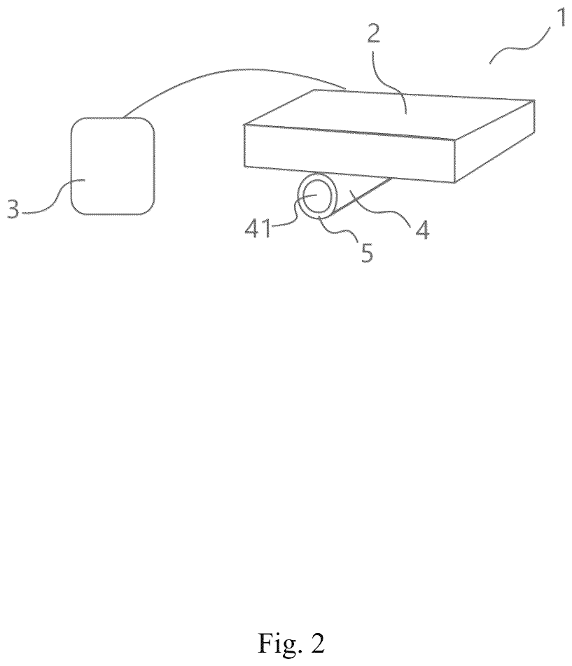

Refer to FIG. 1 and FIG. 2, which are schematic diagrams of an aiming system with different angles. An aiming system 1 includes a positioner 2, an aiming device, and a processor 3. The positioner 2 has a function of acquiring spatial information. The aiming device is connected to the positioner 2, and the processor 3 is connected to the positioner 2 or the aiming device. The positioner 2 includes at least one camera 21 disposed at one side of the positioner 2. The aiming device includes an aimer 4, and the aimer 4 includes at least one hollow channel 41 disposed at a surface of the positioner 2. An axial length of the hollow channel 41 is equal to or greater than a length L1 of the surface of the positioner 2 parallel to an axial direction of the hollow channel 41. In some embodiments, first, FIG. 1 is a schematic diagram of a scanning side of the aiming system (i.e., the side close to the patient's body surface). The aiming system 1 includes the positioner 2, the processor 3 for information transmission and calculation, and the aimer 4. The aimer 4 has the hollow channel 41. More specifically, the scanning side of the aiming system is equipped with a camera of the positioner 2. In this example, the positioner 2 is a dual-lens camera, which includes a camera 21 and a camera 22. It should be understood that the dual-lens camera is one of the embodiments. In some embodiments, a single-lens camera may also be used in the aiming system. Furthermore, multiple sets of lenses may be used to ensure that there are no blind spots in a field of view.

FIG. 2 is a schematic diagram of an alignment side of the aiming system 1 (i.e., the side away from the patient's body surface). The aiming system 1 further includes a prompt signal 5 disposed at one end of the hollow channel 41, and the prompt signal 5 and the camera 21 are located at opposite ends of the positioner 2, respectively. In some embodiments, more specifically, the alignment side of the aiming system 1 has the prompt signal 5.

In addition, information reception and transmission between the components of the aiming system 1 may be through wired transmission or wireless transmission. The schematic diagram of the present disclosure is one of the embodiments. Any conventional information reception/transmission technology can be imported into the present disclosure for use.

### Description of Basic Structure of Aiming System

An aiming system includes a positioner, an aiming device, a processor and a prompt signal, in which the aiming device includes an aimer. A function of the positioner is to acquire the patient's surface information during surgery. The positioner may be any sensing device capable of acquiring spatial information, such as a visible optical sensing device (an RGBD camera, a depth camera, etc.), an infrared optical sensing device, an electromagnetic sensing device, etc. In some embodiments, if the positioner is the visible optical sensing device, it may be a single-lens camera or a multi-lens camera (e.g., two or more lenses).

After the positioner acquires the surface information, the surface information may be transmitted to the processor. At this time, the processor registers the received surface information with the patient's medical image, and calculates a coordinate relationship therebetween and provides coordinate relationship information to the aimer. It should be understood that the surface information acquired by the positioner may be continuous or one-time. In an embodiment of continuously acquiring surface information, the processor may also continuously receive the surface information and calculate, and then continuously update the coordinate relationship and transmit it to the aimer, so that the information acquired by the aimer may be continuously updated in real time. Among them, the processor can receive and transmit the information through wired or wireless methods, and the processor may be externally connected to the positioner or built into the positioner. The schematic diagrams of the embodiments of the present disclosure are for convenience of explanation, so the externally connected processor is shown.

A function of the aimer is to provide a user with a mechanism for aiming and aligning, so that the user can perform alignment outside the body surface without fully exposing a surgical target, and use the aimer to align a surgical instrument with an expected surgical target. The aimer may be a physical aimer or a virtual projection aimer. For example, the aimer may be a physical aimer with a hollow channel, or a virtual aimer presented by holography (or called holographic projection). There may be one or more hollow channels on the aimer, and the hollow channels may be used to provide guidance for different surgical instruments, or to provide guidance for the same surgical instrument at different targets.

In addition, the target aimed at by the aimer can come from surgical planning. All common surgical planning functions in conventional navigation systems may be imported into the present disclosure and used as a basis for surgical planning.

In various embodiments, the aimer and the positioner need to maintain a known spatial relationship. More specifically, the aimer and the positioner may be designed to have a known fixed spatial relationship; or the spatial relationship between the aimer and the positioner must be able to be detected or calculated. It should be noted that a position of the aimer must not interfere with the spatial information sensing of the positioner. For example, if the positioner is a visible optical sensing device, the position of the aimer cannot interfere with or block a field of view of the positioner.

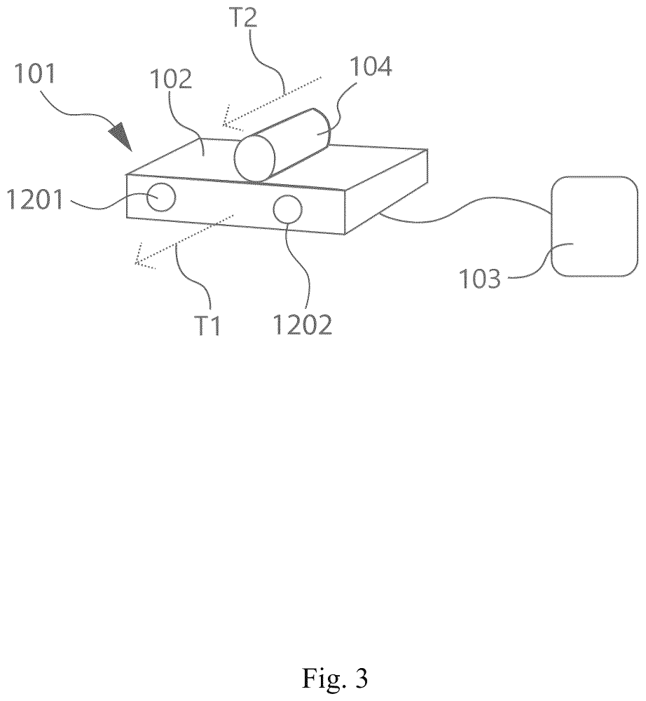

In terms of usage scenario, referring to FIG. 3, an axial direction T2 of an aimer 104 of an aiming system 101 and a sight direction T1 of a sensing device 1201/1202 (e.g., a camera) of a positioner 102 are substantially parallel to each other. In some embodiments, it is a preferred usage scenario that the sight direction T1 of the positioner 102 is parallel to the axial direction T2 of the aimer 104. When the user uses the aimer 104 for alignment, the design allows the positioner 102 to continuously acquire surface information of a target region (and/or its surroundings) and transmit the information to a processor 103 for calculation, and the processor 103 can also continuously update a coordinate relationship between the surface information and a patient's medical image at the same time and then provide to the aimer 104.

What is better in the present disclosure is that all components in the aiming system (including the positioner, the aimer, the hollow channel, the processor, etc.) may be penetrated by x-ray. Specifically, all components in the aiming system are not imaged in an X-ray image. For example, if it is necessary to scan an X-ray image during surgery, all components in the aiming system including the positioner, the aimer, the hollow channel, the processor, etc. are not imaged in the X-ray image, so that the image is not interfered by any component of the aiming system.

### Embodiment 1 of Physical Aimer

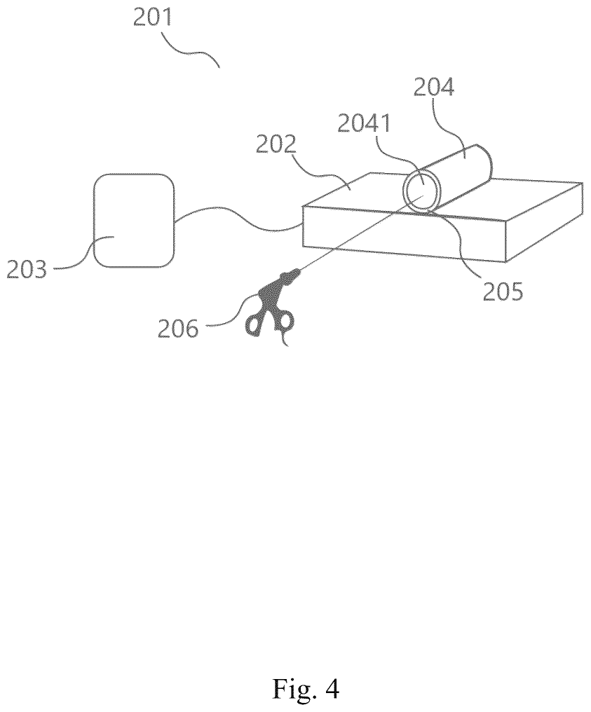

Referring to FIG. 4, the schematic diagram shows a schematic diagram of an alignment side (i.e., the side away from the patient's body surface) of an aiming system 1 in one embodiment. A prompt signal 205 of the aiming system 201 is annular and has a plurality of light sources, and the prompt signal 205 is arranged around along one end of an hollow channel 2041; when a portion of the light sources of the prompt signal 205 emits light, the aiming device moves toward a location where the portion of the light sources are located until the light disappears to complete alignment.

In this embodiment, a positioner 202 and an aimer 204 in the aiming system 201 have a known and fixed spatial relationship, and the fixing method may be any conventional fixing method, including but not limited to engaging, welding, integrally formed, etc. It should be understood that the illustration illustrates one of the fixed spatial relationships, and any fixed spatial relationship may be applied to this embodiment.

In this embodiment, the prompt signal 205 may prompt the user to move the aimer 204 for alignment in different forms. For example, the prompt signal 205 can emit a gradient color light, and the user should move the aimer 204 toward a light with a dark color. When the light emitted by the prompt signal 205 has the same color without gradient, it means that the aimer 204 has moved to the surgical target; or the prompt signal 205 is a plurality of independent light sources, and the user should move the aimer 204 toward a location where the light source is emitted. It should be understood that any method that can provide the user for alignment prompt on the aimer 204 belongs to a form of the prompt signal, and is not limited to the above examples.

When the aimer 204 moves to the surgical target, it means that a position and an angle of the aimer 204 are consistent with surgical planning information. At this time, the user can use a surgical instrument 206 to perform surgery through the hollow channel 2041 of the aimer 204. The surgical instrument 206 may be any instrument or tool used in the surgery, and is not limited to the example shown in the schematic diagram of this embodiment.

Furthermore, in this embodiment, if the user needs, a display may also be connected to the aimer 204. The display is used to display any information about the surgery, including but not limited to alignment prompt Information, medical imaging information, surgical planning, etc. The above information may be calculated by the processor and provided to the display for display. In this design, the display is detachable and may be connected to the aimer 204 for use, or may be detached and used elsewhere.

### Embodiment 2 of Physical Aimer

In some embodiments, an aimer may be combined with a display, so that the aimer has a function of displaying image information or figure information. An advantage of this design is that it can display a medical image of the patient's body surface range and/or its surrounding corresponding to the aimer at that time based on a spatial relationship calculated by a processor. The medical image includes but is not limited to a visible light image of the body surface range and/or its surroundings, a fluoroscopic medical image of the body surface range and/or its surroundings, etc.

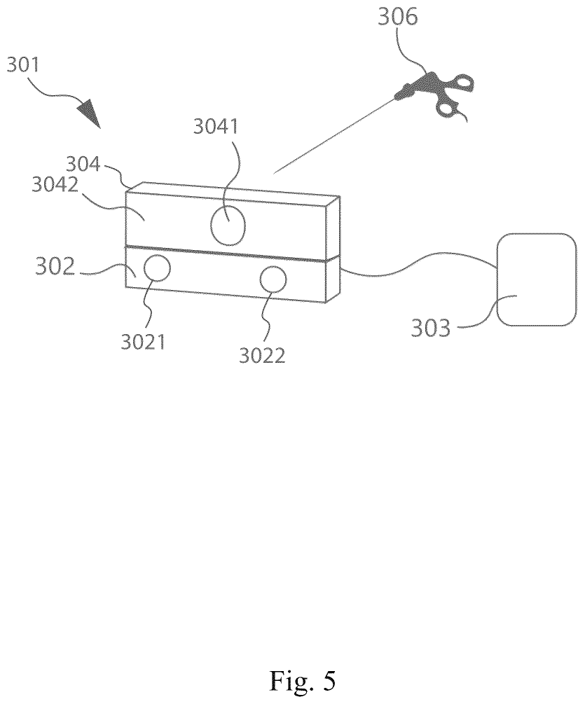

Referring to FIG. 5, the schematic diagram shows a schematic diagram of a scanning side (i.e., the side close to the patient's body surface) of an aiming system 301 in one embodiment. A display 3042 of the aiming system 301 is disposed on a positioner 302, and an aimer 304 is penetrated through the display 3042. The aimer 304 itself is a display with a hollow channel 3041 in the middle. In other words, the aimer 304 is the display 3042 with the hollow channel 3041. In this embodiment, the aimer 304 and the positioner 302 have a known and fixed spatial relationship, and the fixing method may be any conventional fixing method, including but not limited to engaging, welding, integrally formed, etc. It should be understood that the illustration illustrates one of the fixed spatial relationships, and any fixed spatial relationship may be applied to this embodiment.

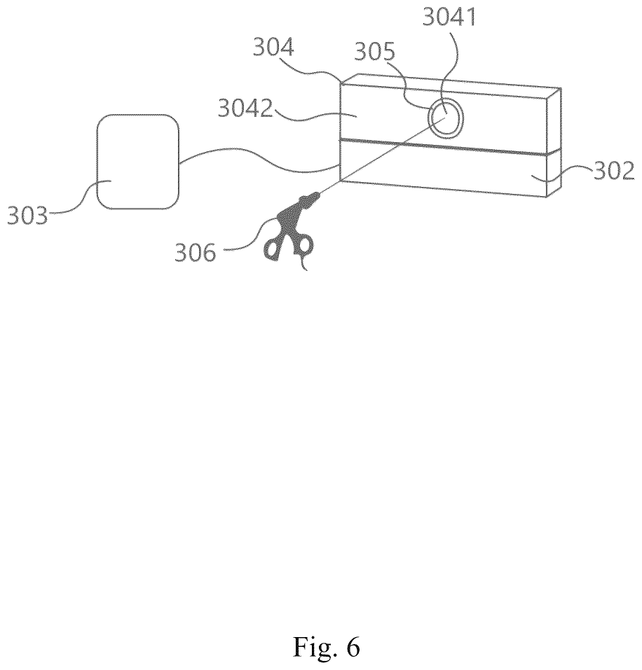

Referring to FIG. 6, the schematic diagram shows a schematic diagram of an alignment side (i.e., the side away from the patient's body surface) of the aiming system 301 in the same embodiment as that of FIG. 5. The aiming system 301 further includes a prompt signal 305 disposed at one end of the hollow channel 3041, and the prompt signal 305 and the sensing devices 3021/3022 of the positioner 302 are respectively located at opposite ends of the positioner 302. Similar to the alignment signal prompt in the other embodiments mentioned above, the user can move the aimer 304 for alignment according to different forms of the prompt signal 305, such as the gradient light, the independent light sources, etc. mentioned in the above-mentioned embodiments, and when the aimer 304 moves to the surgical target, it means that the position and the angle of the aimer 304 are consistent with the surgical planning information. At this time, the user can use the surgical instrument 306 to perform surgical treatment through the aimer 304. In addition, in this embodiment, the display 3042 (e.g., an image display panel) may also be provided on an image display side (i.e., the side of the aimer away from the patient's body surface) of the aimer 304. In addition to using the prompt signal 305 to prompt for alignment, the alignment prompt information may be directly superimposed on the image displayed on the display 3042.

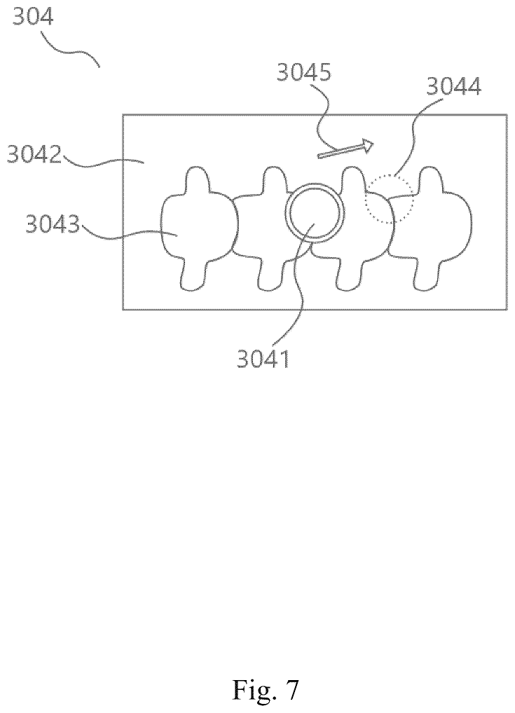

In order to present the schematic diagram of the embodiment on the display 3042 of the aimer 304 more clearly, FIG. 7 only enlarges the portion of the schematic diagram. In this embodiment, in addition to displaying a patient's medical image 3043, the display 3042 on the aimer 304 may also present any other information needed during the surgery, including but not limited to a guidance alignment prompt, basic patient information, surgery planning information, screen of a sight range of the positioner, etc.

For example, when the hollow channel 3041 on the aimer 304 is not aligned with a surgical plan 3044, a guidance alignment prompt 3045 appears on the display 3042 to prompt the user in a direction in which the aimer 304 should be moved toward. When the user accurately aligns the position and the angle of the hollow channel 3041 on the aimer 304 with the surgical plan 3044, the display 3042 may display accurate alignment prompt information.

It should be understood that the prompt information on the display 3042 is not limited to any shape, size, type, color, etc., as long as presentation of different status prompts is displayed in a visual form, those can be presented on the display 3042. In addition, the display 3042 may also provide different sensory prompts, including but not limited to vibration, a sound prompt, etc.

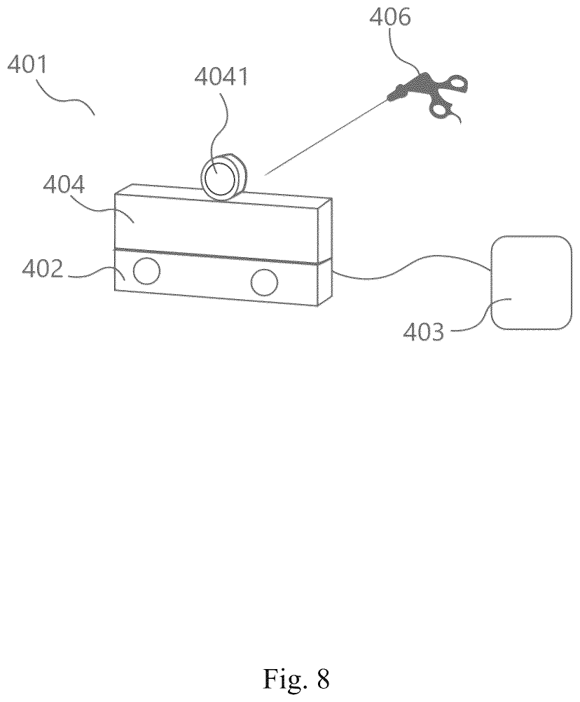

Referring to FIG. 8, in another embodiment in which the aimer is a display with a hollow channel, and the hollow channel does not need to be in the middle of the display, but is fixed on the display. The fixing method may be any conventional fixation method. The fixing method includes but is not limited to engaging, welding, integrally forming, etc. It should be understood that the illustration illustrates one of the fixed spatial relationships, and any fixed spatial relationship may be applied to this embodiment.

As shown FIG. 8, an aiming system 401 is equipped with a positioner 402, a processor 403, and an aimer 404, in which the aimer 404 is composed of a display and a hollow channel 4041 fixed on the display. It should be understood that this schematic diagram shows a different type of the aimer 404. The embodiments corresponding to FIGS. 5 to 7 may be applied to the embodiment of the aimer type of FIG. 8.

Furthermore, in the embodiment where the aiming system is equipped with the display, the display may be designed to have a function of switching a display content. For example, one or more of a guidance alignment prompt, a medical image, surgical planning, screen of a sight range of the positioner, other surgical information, etc. may be selectively displayed. All displayed images may be turned off when needed, and thus the display appears transparent.

### Embodiment of Virtual Aimer

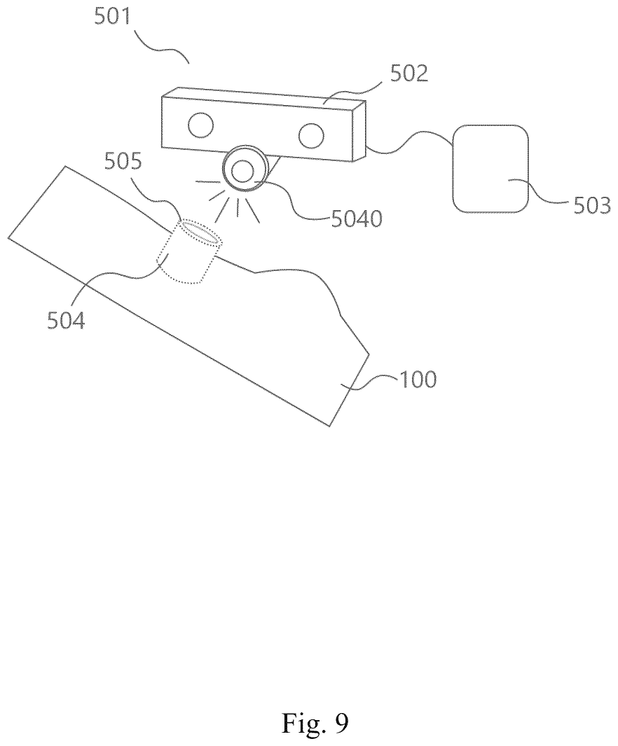

Referring to FIG. 9, in some embodiments, the aimer may be a virtual aimer presented through projection. More specifically, a projection method may be holography (or called holographic projection). An aiming device of an aiming system 501 includes a projection light source 5040, an aimer 504 (virtual aimer), a prompt signal 505 (a virtual prompt signal), and surgical information (virtual surgical information). The projection light source 5040 is disposed at a surface of a positioner 502, and the virtual aimer 504 is projected and displayed by the projection light source 5040. The virtual prompt signal 505 is projected and displayed by the projection light source 5040, and the virtual prompt signal 505 is presented at one end of the virtual aimer 504. The virtual surgical information is projected and displayed by the projection light source 5040, and the virtual surgical information is presented at one side of the virtual aimer 504, including but not limited to below.

FIG. 9 illustrates one of embodiments of holography (or called holographic projection). In this embodiment, an aiming system 501 includes a positioner 502, a processor 503, an aimer 504, a prompt signal 505 and a projection light source 5040, in which the aimer 504 and the prompt signal 505 are virtual images projected through the projection light source 5040. After the positioner 502 acquires surface information of the patient 100, the surface information is transmitted to the processor 503 to register the surface information with the patient's medical image and calculate a coordinate relationship between the two, and then provide coordinate relationship information to the projection light source 5040, and the projection light source 5040 then projects surgical information in a field of view of the positioner 502 at that time based on the coordinate relationship. The surgical information includes at least one of surgical planning in the field of view, a patient medical imaging perspective view, other surgical information, a surgical prompt, screen of a sight range of the positioner, etc. The surgical prompt information may include all visual prompt information and alignment guidance mentioned in the above embodiments, and presentation of the surgical prompt information may be a projected 3D image or a 2D image.

In addition, in the embodiment, the projection light source 5040 should maintain a known spatial relationship with the positioner 502. More specifically, the projection light source 5040 and the positioner 502 may be designed to have a known fixed spatial relationship; or the spatial relationship between the projection light source 5040 and the positioner 502 must be able to be detected or calculated.

It should be understood that holography (or called holographic projection) has different forms of the projection light source, and any form of the projection light source may be incorporated into the present disclosure and is not limited to the light source shown in this schematic diagram.

### Embodiments of Use of Aiming System with Other Accessories

In order to be friendlier to use, the aiming system may be used with other accessories. The following embodiments illustrate several main accessories.

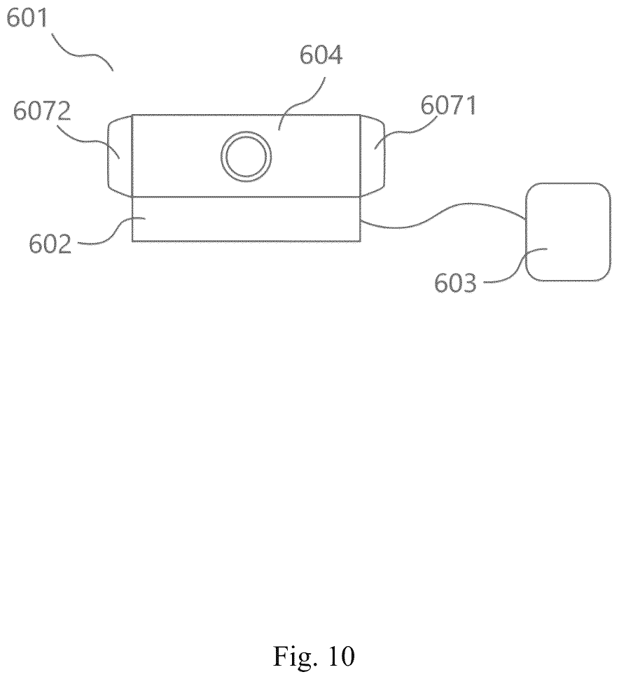

Referring to FIG. 10, an aiming system 601 further includes at least one handle 6071 disposed at one side of a positioner 602 or an aimer 604. In some embodiments, the aiming system 601 includes a positioner 602, a processor 603, an aimer 604, and handles 6071 and 6072. A function of the handles 6071 and 6072 is to allow the user to hold more conveniently when operating the aiming system 601 without affecting a field of view of the positioner 602, and also to prevent the user from blocking a sight due to improper holding when operating the aiming system 601. The handles 6071 and 6072 may be used in conjunction with any of the above embodiments.

It should be understood that a form and a position of the handle are not limited to the schematic diagram of this embodiment. The handles with any known shape, size, material, and type may be fixed at the aiming system to achieve the same effect.

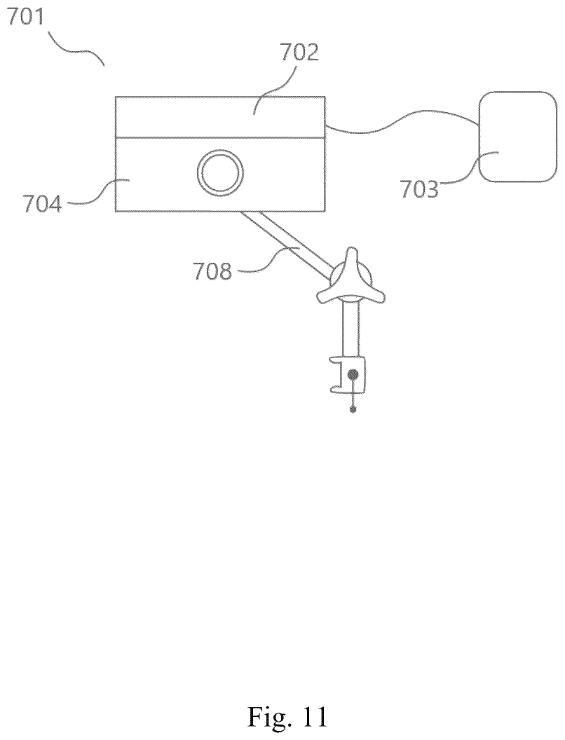

Referring to FIG. 11, an aiming system 701 further includes a connecting rod 708 disposed beneath a positioner 702 or an aiming device (e.g., an aimer 704), so that the aiming system 701 is locked and fixed in space with any posture. In some embodiments, the aiming system 701 includes a positioner 702, a processor 703, an aimer 704, and a supportable connecting rod 708. The connecting rod 708 is used to provide a supporting force for the aiming system 701 to allow the user to lock the aiming system 701 in space with any posture more efficiently and with less effort when operating the aiming system 701.

In this embodiment, the connecting rod 708 may be any form of a support arm, including but not limited to electric, manual, pneumatic, etc. In addition, the connecting rod 708 may be connected to any position of the aiming system. More specifically, it may be connected to the positioner 702 or the aimer 704 to achieve effects of reducing a weight on the user's hand and stabilizing the aiming system 701. The connecting rod 708 may be combined with any of the above embodiments.

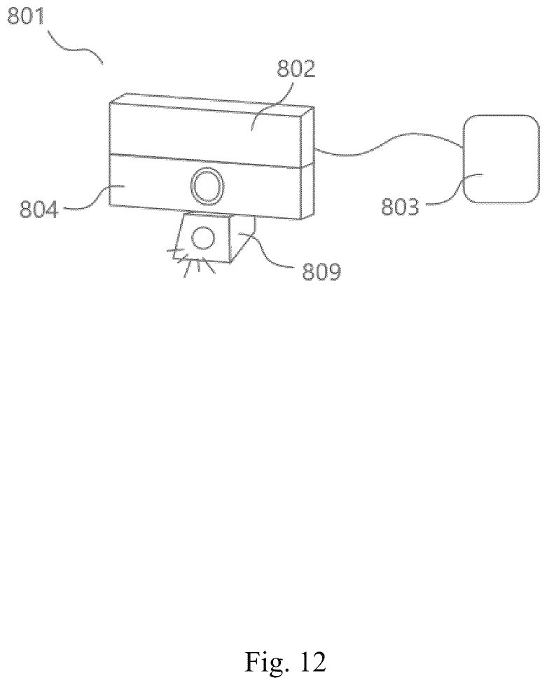

Referring to FIG. 12, an aiming system 801 further includes a visible light source 809 disposed at one side of a positioner 802 or one side of an aiming device (e.g., an aimer 804). In some embodiments, the aiming system 801 includes a positioner 802, a processor 803, an aimer 804, and a visible light source 809. A function of the visible light source 809 is that when the aiming system 801 is used during surgery, operating light in the operating room may be blocked due to the position or the angle of the aiming system 801, so that the light in the operating room may need to be adjusted frequently during the surgery; or the light in the operating room is blocked, and thus affects a visual effect of the aiming system 801. The visible light source provided on the aiming system 801 allows the user to have a more sufficient light source to perform the surgery with focus. If a visible light sensing device is used in the positioner 802 in the aiming system 801, the visible light source 809 may achieve the lighting effect simultaneously, so that the positioner 802 can have better vision and light, thereby improving a subsequent imaging effect. Furthermore, the visible light source 809 may be designed as a controllable light source, including but not limited to one or more of control of a direction of the light source, intensity, hue, etc. The visible light source 809 may be combined with any of the above embodiments.

In some embodiments, regarding a fixed condition (mechanism parameters are fixed and known), the positioner 802 and the aimer 804 are non-coaxial (off-centre). If there are a plurality of positioners 802, those are all non-coaxially (off-centre) arranged with the aimer 804. Regarding a movable condition (the mechanism parameters may be variable and known), the above principles remain unchanged, and the positioner 802 may be designed to be movable and the aimer 804 to be immobile; or the positioner 802 may be designed to be immobile and the aimer 804 to be movable; or both may be designed to be movable. As for a coordinate update method, if it is the fixed condition, it is an original design value. If it is the movable condition, updated relative coordinates may be known through an encoder on a slide table or a slide rail; or a plurality of fixed known positions may be designed, and a switch mechanism may be used to know the position and acquire the relative coordinates.

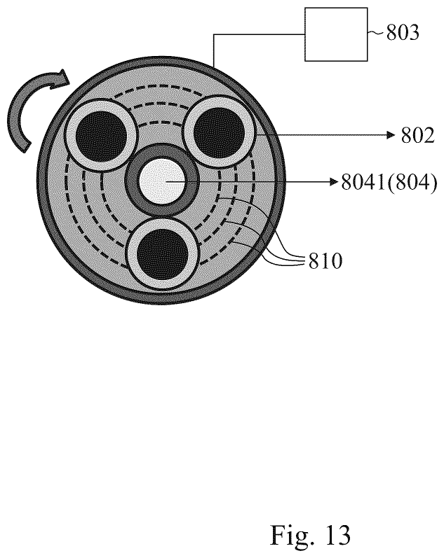

Please refer to FIG. 13, which is a schematic diagram of non-coaxial arrangement of a movable positioner and an immobile aimer of an aiming system of some embodiments of the present disclosure. An aiming system 801 includes a positioner 802, a processor 803, an aimer 804 and a plurality of slide grooves 810. The aimer 804 includes a hollow channel 8041, and the slide grooves 810 are sequentially arranged around outside the hollow channel 8041. The positioner 802 includes a plurality of sensing devices respectively movably disposed in the slide grooves 810 and movably arranged around the aimer 804. Specifically, the positioner 802 is movable. When encountering a shielding issue, the positioner 802 may be adjusted to a suitable positioning posture.

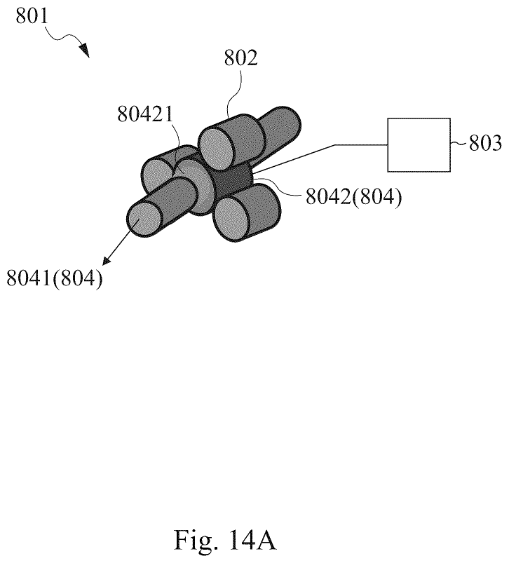

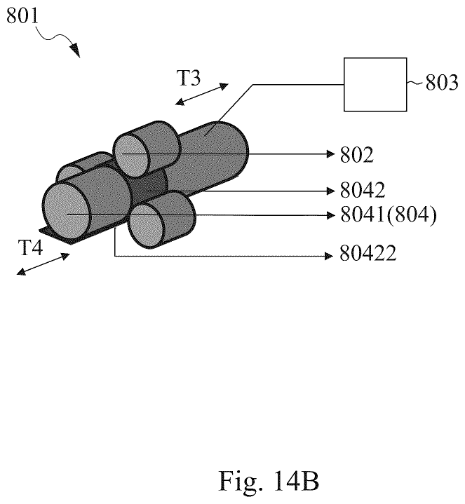

Please refer to FIG. 14A, which is a schematic diagram of non-coaxial arrangement of an immobile positioner and a rotatable movable aimer of an aiming system of some embodiments of the present disclosure. An aiming system 801 includes a positioner 802, a processor 803, and an aiming device (an aimer 804). The aimer 804 includes a base 8042 and a hollow channel 8041 movably penetrated through the base 8042. The positioner 802 includes a plurality of sensing devices respectively disposed at an outer surface of the base 8042. The base 8042 includes a ball joint 80421, and the hollow channel 8041 is rotatably movably penetrated through the ball joint 80421.

Please refer to FIG. 14B, which is a schematic diagram of non-coaxial arrangement of an immobile positioner and an aimer that is forwardly and backwardly movable of an aiming system of some embodiments of the present disclosure. The difference from FIG. 14A is that the base 8042 includes a slide rail 80422, and an axial direction T3 of a hollow channel 8041 is parallel to an axial direction T4 of the slide rail 80422, and the hollow channel 8041 is movably penetrated through the base 8042 along the axial direction T4 of the slide rail 80422.

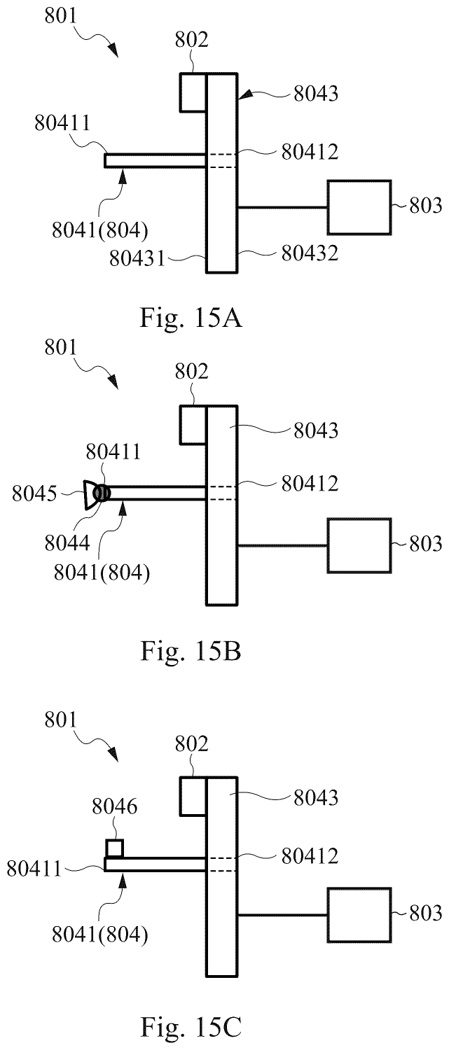

Please refer to FIG. 15A, which is a schematic diagram of an aimer combined with a display and a positioner of an aiming system of some embodiments of the present disclosure. An aiming system 801 includes a positioner 802, a processor 803, an aimer 804 and a display 8043. The display 8043 includes a first side 80431 and a second side 80432 opposite to the first side 80431. The aimer 804 includes a hollow channel 8041 disposed on the display 8043 or penetrated through the display 8043. The hollow channel 8041 has a first end 80411 and a second end 80412 opposite to the first end 80411. The second end 80412 and a second side 80432 of the display 8043 are coplanar with each other. The positioner 802 is disposed on the display 8043. In some embodiments, the aimer 804 may be additionally connected to another aiming display 8043 having a plurality of hollow channels 8041. The aiming display 8043 may be customized according to a planned path. For example, a display 8043 may be customized specifically for patient's surgical needs, and the display 8043 has a hollow channel 8041 suitable for the patient's surgical path.

Please refer to FIG. 15B, which is a schematic diagram of an aimer combined with a display, a positioner and a movable interface of an aiming system of some embodiments of the present disclosure. The difference from FIG. 15A is that the aimer 804 further includes a ball joint 8044 and a suction cup 8045. The ball joint 8044 is disposed at the first end 80411 of the hollow channel 8041. The suction cup 8045 is disposed at a side of the ball joint 8044 opposite to the first end 80411, that is, the ball joint 8044 is between the suction cup 8045 and the first end 80411 of the hollow channel 8041. Specifically, a front end of the aimer 804 may be combined with the suction cup 8045 and the ball joint 8044, which may be used as a fulcrum for rotational adjustment and alignment.

Please refer to FIG. 15C, which is a schematic diagram of an aimer combined with a display, a positioner and a sensor of an aiming system of some embodiments of the present disclosure. The difference from FIG. 15A is that the aimer 804 further includes a sensor 8046 disposed at the first end 80411 of the hollow channel 8041. Specifically, the sensor 8046 may be placed at the front end of the aimer 804, which can initiate a signal when touching an object. For example, the sensor 8046 is a piezoelectric/thin film sensor that can detect a touch when touching the object to generate the signal.

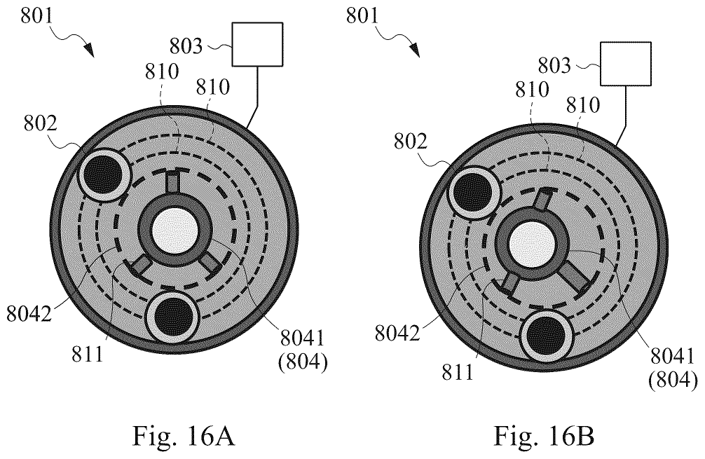

Please refer to FIGS. 16A and 16B, which are schematic diagrams illustrating operations of an aiming system with a fine-tuning mechanism of some embodiments of the present disclosure. An aiming system 801 includes a positioner 802, a processor 803, and an aiming device. The aiming device includes an aimer 804, a base 8042, a plurality of slide grooves 810, and a fine-tuning mechanism 811. The base 8042 is hollow ring-shaped, and the slide grooves 810 are sequentially arranged around an outer wall of the base 8042. The aimer 804 includes a hollow channel 8041, and the fine-tuning mechanism 811 is disposed between an outer wall of the hollow channel 8041 and an inner wall of the base 8042, so that the hollow channel 8041 may be movably penetrated through the base 8042. Specifically, there may be one or more fine-tuning mechanisms 811, such as three fine-tuning mechanisms 811. The fine-tuning mechanism 811 is used to automatically push an instrument onto a target path within a compensable range.

In some embodiments, the positioner 802 may be a color camera (RGB camera), a color depth camera (an RGBD camera), or other stereo positioning technology (e.g., structured light, time-of-flight (ToF), radio frequency (RF)), etc. In some embodiments where the positioner 802 uses a dual camera, a base of the positioner 802 is a variable base, and a base structure may be a spherical platform or a parallel robot platform to allow the positioner 802 to perform different actions depending on the situation. Scenario 1: since it is necessary to consider evaluation and data collection of surrounding environment as much as possible during setting up the positioner 802, the base can turn the positioner 802 outward, so that a field of view is as wide as possible. Scenario 2: during surgery, stereo vision is required for positioning, so the base can turn the positioner 802 inward to increase a range of a stereo vision area. If there are multiple cameras or multiple consecutive stereoscopic images, a target object (including a target or an obstacle) in an aiming scene may be extracted and post-processed, including erasure, color change, etc. If there are multiple cameras or multiple consecutive stereoscopic images, a predetermined behavior of the aimer 804 may be updated and practiced in virtual reality, allowing the user to feel and evaluate effectiveness and safety of the method.

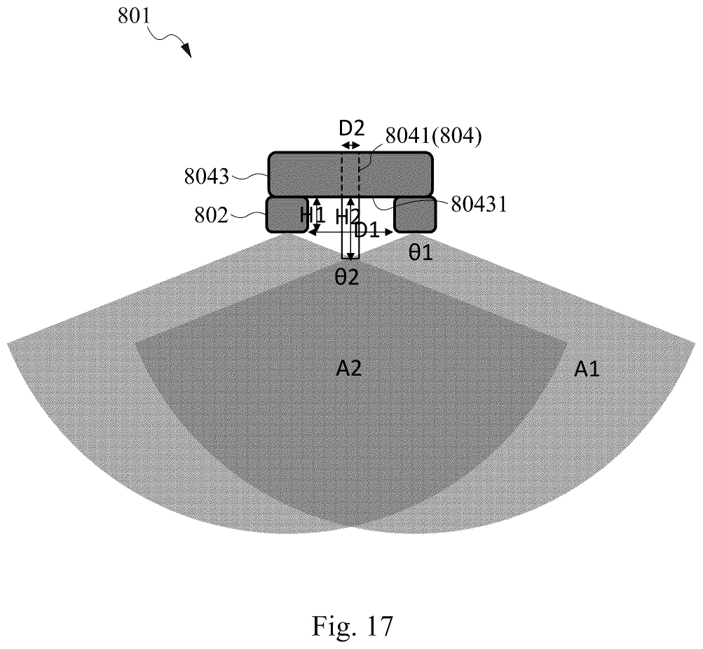

Please refer to FIG. 17 for details, which is a schematic diagram of a dual positioner, an aimer and a display of an aiming system of some embodiments of the present disclosure. The difference between FIG. 17 and FIG. 15A is in the viewing angle. The positioner 802 protrudes from a surface of the display 8043 by a first height H1, and the hollow channel 8041 protrudes from a surface of the display 8043 by a second height H2. The first height H1 is smaller than the second height H2. The positioner 802 includes two sensing devices (e.g., cameras) respectively disposed at both ends of the first side 80431 of the display 8043, and there is a distance D1 between the two sensing devices, and the hollow channel 8041 has a diameter D2, and the diameter D2 is less than or equal to the distance D1. Specifically, the second height H2 minus the first height H1 should be as small as possible (Similarly, a camera viewing angle 81 should be as large as possible. If there are two cameras set in parallel, an overlapping viewing area of the two cameras has an angle 82, and the angle 81 and the angle 82 are substantially the same). If the aimer 804 itself does not have a depth sensing mechanism, a preset length of the aimer 804 should be greater than or equal to H2 in order to be tracked. If it shrinks to less than H2 due to circumstances, coordinates need to be confirmed through an encoder. For example, if there is a displacement sensor or another encoder in the hollow channel 8041, coordinates of an instrument before entering a field of view (FOV) of the positioner 802 may be known. A stereotaxic range is area A2. Area A1 is a visible range of a single camera minus the area A2, which is not a stereotaxic range.

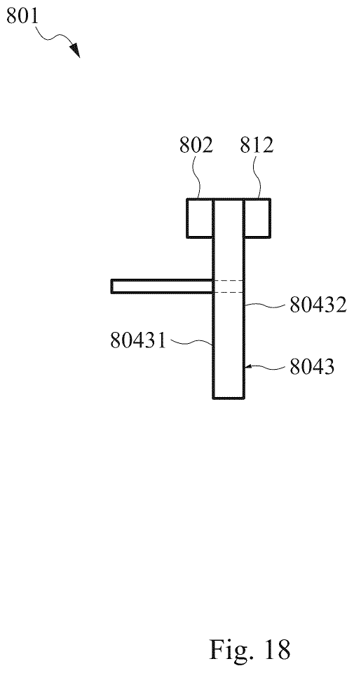

Please refer to FIG. 18, which is a schematic diagram of an aiming system with an observer according to some embodiments of the present disclosure. The difference from FIG. 15A is that the aiming system 801 further includes an observer 812. The positioner 802 is disposed on the first side 80431 of the display 8043, and the observer 812 is disposed on the second side 80432 of the display 8043 (e.g., close to the user end) to observe user behavior. Specifically, a setting direction of the observer 812 may be different from a direction of the positioner 802. A commonly used aspect is an opposite direction of the positioner 802. The observer 812 is used to observe the user behavior, such as (1) eye tracking: it can track the user's gaze and transmit a prompt signal or a guidance prompt screen into a mode that is more convenient for the user to view; (2) gesture control: the user can control the system non-contactly through gestures; (3) reminder of user error: the user behavior when operating the system may be observed, and a warning is issued if there is an error in prediction; (4) instrument identification: it can identify whether the instrument to be used with the system meets the settings, if not, a warning is issued; (5) surgical process observation and system process automatic switching: it can observe the user's surgical steps and predict and assist in quick process switching to save time and redundant operations. The observer 812 may also be the positioner 802 at the same time. If both ends are the positioner 802 and the observer 812, a bidirectional aiming/alignment function may be implemented. Furthermore, the aiming system 801 may be combined with a light source, and a front end of the aimer 804 may be combined with a light source to form a surgical light effect. Uniformizing the light in an area to be operated on facilitates camera identification and tracking.

In some embodiments, the aimer 804 may be retractable. The aimer 804 may be a traction handle. In some embodiments, the aimer 804 incorporates an instrument clamping mechanism. The clamping effect of the mechanism may be either clamping or relaxing. The clamping effect of the mechanism is variable. The more accurate the alignment, the tighter it is, and the farther out the center is, the more flexible it is. In some embodiments, the aimer contains a matching fixing mechanism, which may be a common matching feature of the instrument (e.g., D-shaped, square-shaped, hexagonal-shaped, contouring, etc.). It should be understood that the embodiments corresponding to FIGS. 13 to 18 may be applied to the aimer and its additional technical features of the embodiments described here.

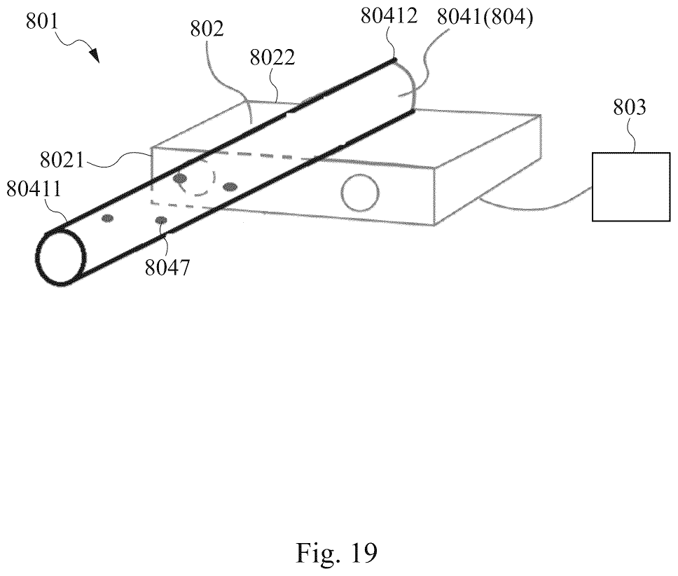

Please refer to FIG. 19, which is a schematic diagram of an aiming system with calibration features at an aimer according to some embodiments of the present disclosure. An aiming system 801 includes a positioner 802, a processor 803, and an aiming device (including an aimer 804). The positioner 802 includes a first side 8021, a second side 8022 opposite to the first side 8021, and at least one camera disposed at the first side 8021. The aimer 804 includes a hollow channel 8041 and calibration features 8047. The hollow channel 8041 is disposed on the positioner 802. The hollow channel 8041 has a first end 80411 and a second end 80412 opposite to the first end 80411. The second end 80412 is coplanar with the second side 8022 of the positioner 802. The first end 80411 of the hollow channel 8041 protrudes from the first side 8021 of the positioner 802. The calibration features 8047 are disposed at the first end 80411 of the hollow channel 8041, and a distance between the calibration features 8047 and the first side 8021 of the positioner 802 is greater than or equal to a shortest focal length of the at least one camera. Specifically, the aimer 804 is used as a calibration medium. The aimer 804 has a certain length (e.g., twice the shortest focal length). In some embodiments, the length of the aimer 804 is 1 micron to 100 centimeters; in some embodiments, the length of the aimer 804 may be 7 centimeters to 70 centimeters. The positioner 802 may directly identify the calibration features 8047 on the aimer 804, and a number of the calibration features 8047 may be increased to enhance identification and avoid problems such as light intensity or occlusion affecting the identification during operation. Furthermore, when the calibration features 8047 are completely identified, an orientation of the aimer 804 may be calibrated.

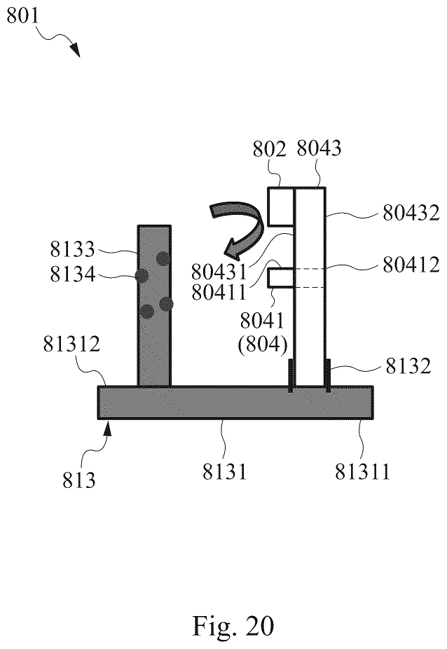

Please refer to FIG. 20, which is a schematic diagram of an aiming system with a calibration block of some embodiments of the present disclosure. An aiming system 801 includes a positioner 802, a processor 803, an aiming device (including an aimer 804) and a calibration device 813. The calibration device 813 includes a base 8131, a combination base 8132 and a bump 8133. The base 8131 has a first end 81311 and a second end 81312 opposite to the first end 81311. The combination base 8132 is disposed at the first end 81311 of the base 8131, and one end of the display 8043 is detachably combined with the combination base 8132. The bump 8133 protrudes from the second end 81312 of the base 8131. The bump 8133 has calibration features 8134, and the calibration features 8134 are located in front of the first end 80411 of the hollow channel 8041, in which the positioner 802 is movably disposed on the first side 80431 of the display 8043 to perform calibration according to positions of the calibration features 8134. Specifically, the positioner 802 is used as a calibration medium, and there are known features (the calibration features 8134) on the bump 8133 in the calibration device 813 (calibration block), and the positioner 802 is used to identify the features. The positioner 802 makes a known movement or rotation to perform calibration.

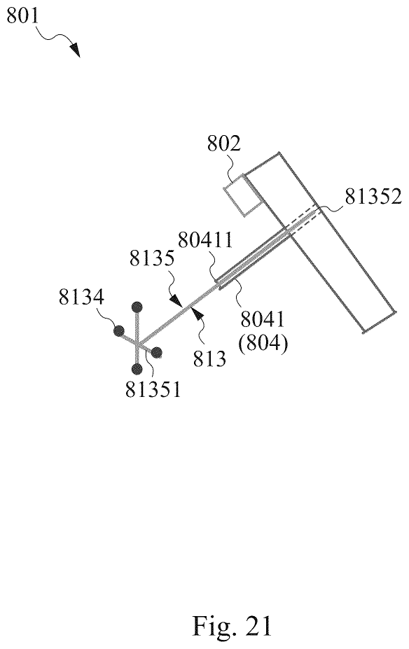

Please refer to FIG. 21, which is a schematic diagram of an aiming system with a calibration rod of some embodiments of the present disclosure. The difference from FIG. 20 is that the calibration device 813 includes a rod body 8135 and calibration features 8134. The rod body 8135 has a first end 81351 and a second end 81352 opposite to the first end 81351. The second end 81352 is detachably penetrated through and in the hollow channel 8041 or sleeved outside the hollow channel 8041. The calibration features 8134 are disposed at the first end 81351 of the rod body 8135, and the calibration features 8134 are located in front of the first end 80411 of the hollow channel 8041, in which the hollow channel 8041 is axially moved or rotated according to positions of the calibration features 8134 for calibration, and the rod body 8135 may also be axially moved or rotated along the hollow channel 8041 for calibration. Specifically, the calibration rod is used as a calibration medium, and the rod body 8135 (calibration rod) is designed and inserted from a front end of the aimer 804, and may be placed at an inner diameter of the aimer 804 or sleeved on an outer diameter of the aimer 804 to move or rotate along the axial direction for calibration.

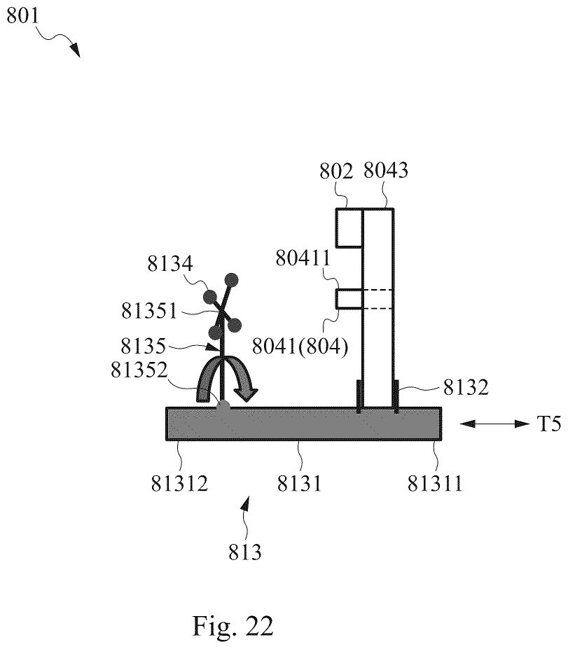

Please refer to FIG. 22, which is a schematic diagram of an aiming system with a calibration block and a calibration rod of some embodiments of the present disclosure. The difference from FIG. 21 is that the calibration device 813 includes a base 8131, a combination base 8132, a rod body 8135 and calibration features 8134. The base 8131 has a first end 81311 and a second end 81312 opposite to the first end 81311. The combination base 8132 is disposed at the first end 81311 of the base 8131, and one end of the display 8043 is detachably combined with the combination base 8132. The rod body 8135 has a first end 81351 and a second end 81352 opposite to the first end 81351. The second end 81352 of the rod body 8135 slides or rotates along an axial direction T5 of the base 8131. The calibration features 8134 are disposed at the first end 81351 of the rod body 8135, and the calibration features 8134 are located in front of the first end 80411 of the hollow channel 8041, in which the positioner 802 is fixed and may be calibrated by sliding and rotating the calibration features 8134 on the rod body 8135. Specifically, the calibration device 813 (calibration block) is used with the rod body 8135 (calibration rod), and the calibration rod is connected to the calibration block, and the calibration rod is moved and rotated through the existing slide grooves on the calibration block for calibration.

### Embodiments of Hollow Channel on Aimer

The hollow channel on the aimer is used to guide the user to the position of the surgical target, so its accuracy is extremely important. The above-mentioned processor can continuously receive the surface information and perform calculation, and then continuously update the coordinate relationship information to the aimer, so that the information acquired by the aimer may be continuously updated in real time. Another key point is a matching degree between the hollow channel and the instrument. The following embodiments illustrate the relationship between the instrument and the hollow channel. In order to more clearly present the key point to be described in the paragraphs, figures of the embodiments in the paragraphs only enlarge the aimer, the hollow channel and the instrument.

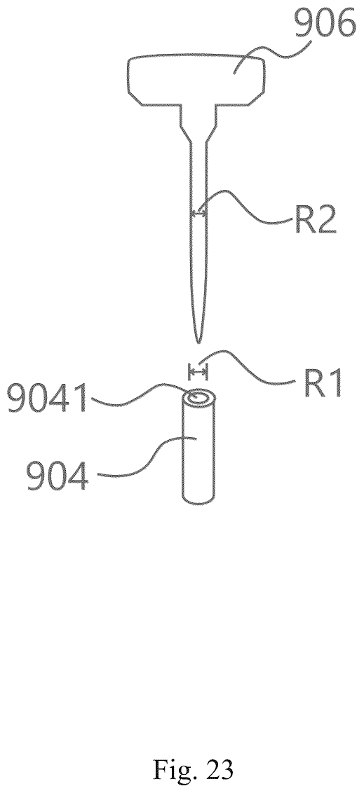

Referring to FIG. 23, the aiming device includes an aimer 904, which includes a hollow channel 9041 disposed at one side of the positioner, in which an inner diameter R1 of the hollow channel 9041 is greater than or equal to an outer diameter R2 of the instrument 906. In this embodiment, the instrument 906 and the hollow channel 9041 of the aimer 904 are matched with each other through structures with contoured shape. More specifically, the outer diameter R2 of the surgical instrument 906 and the inner diameter R1 of the hollow channel 9041 are in a matching relationship. The surgical instrument 906 includes but is not limited to a surgical dilator, a puncture needle, an endoscope, an ultrasonic probe, a screw driver, etc.

In addition to the hollow channel 9041 itself designed to match the shape of the instrument 906 as described in the above embodiments, the structure to achieve contour matching may be achieved through other methods to achieve effects of contour matching and fixing the instrument. The aimer 904 further includes an inflatable structure disposed at an inner wall of the hollow channel 9041. For example, an inner wall of the hollow channel 9041 may be provided with the inflatable structure. When the instrument 906 enters the hollow channel, the inflation function is turned on. At this time, the instrument 906 will be covered and fixed in the hollow channel 9041 by the inflatable structure.

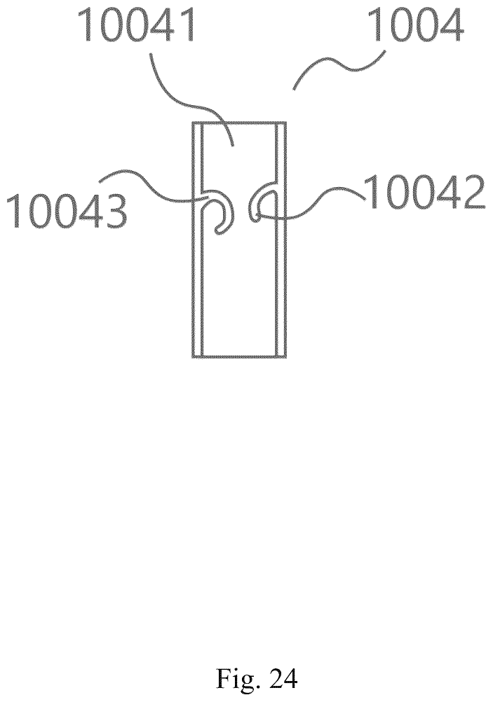

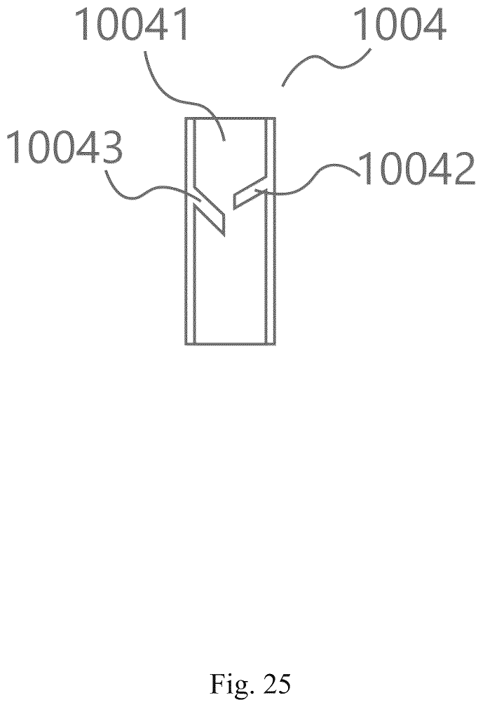

Or referring to FIG. 24, the schematic diagram shows a cross-sectional view of an aimer 1004. The aimer 1004 further includes at least one vacuum feature 10042 disposed at an inner wall of the hollow channel 10041. When the at least one vacuum feature 10042 is in a vacuum state, the at least one vacuum feature 10042 is in a hardened state. When the at least one vacuum feature 10042 is in a non-vacuum state, the at least one vacuum feature 10042 is in a soft state. In some embodiments, the inner wall of the hollow channel 10041 has two vacuum features 10042 and 10043. When the instrument extends into the hollow channel 10041, the vacuum function may be turned on, so that the vacuum features 10042 and 10043 on the inner wall of the hollow channel 10041 harden to achieve the effect of stabilizing the instrument. FIG. 25 illustrates an effect of the vacuum features 10042 and 10043 after vacuum hardening. More specifically, before the vacuum features 10042 and 10043 are evacuated, the vacuum features 10042 and 10043 are soft structures, and harden after being evacuated.

It should be understood that FIG. 24 is only a situation illustrating the embodiment. The vacuum feature may be one or more, and may be any shape, size, etc., and is not limited to the illustration of this embodiment.

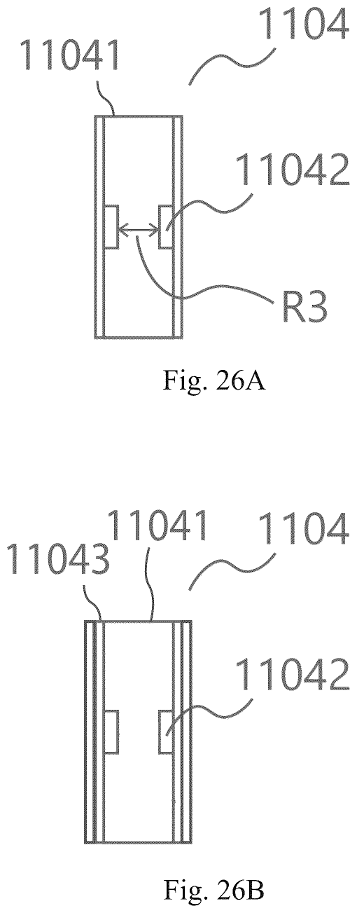

Furthermore, an adapter may be provided between the instrument and the hollow channel. Referring to FIG. 26A, the aimer 1104 further includes an adapter 11042. The adapter 11042 is annular and is disposed at the inner wall of the hollow channel; an inner diameter R3 of the adapter 11042 is greater than or equal to an outer diameter of the instrument. This embodiment is a cross-sectional view of the aimer 1104 with the adapter 11042. The adapter 11042 is a ring-shaped structure, and the adapter 11042 also has above functions of contouring, inflating, and vacuuming. The ring-shaped structure may be a dilator with an outer diameter smaller than an inner diameter of the aimer 1104, or the ring-shaped structure composed of a plurality of collars with an outer diameter smaller than the inner diameter of the aimer 1104, or may be in the form of a clamping jaw with at least three claws, and the clamping jaws are evenly distributed on the ring-shaped structure. The ring-shaped structure needs to fit into the aimer 1104. More specifically, the inner diameter R3 of the adapter 11042 may be designed to match the diameter of the instrument, or the adapter 11042 itself has the function of inflating or vacuuming, so that the adapter 11042 can match and fix the instrument. In addition, the way in which the adapter 11042 is connected to the inner wall of the hollow channel 11041 may be in any form, including but not limited to magnetic attraction, engaging, integrally formed, etc.

Further, please refer to FIG. 26B. The aimer 1104 further includes a dedicated drape 11043 disposed between the hollow channel 11041 and the adapter 11042. In some embodiments, the adapter 11042 may be a component that is fused and fixed on the dedicated drape 11043, or the adapter 11042 and the inner wall of the hollow channel 11041 are fixed so that the dedicated drape 11043 can be sandwiched between the two, and the advantage of this approach is that it can establish a surgical sterile surface.

In addition to ensuring the matching and stability between the instrument and the hollow channel, another key point during use is to let the user know a current depth of the instrument in the hollow channel to ensure that the instrument will not cause unexpected harm due to being too deep.

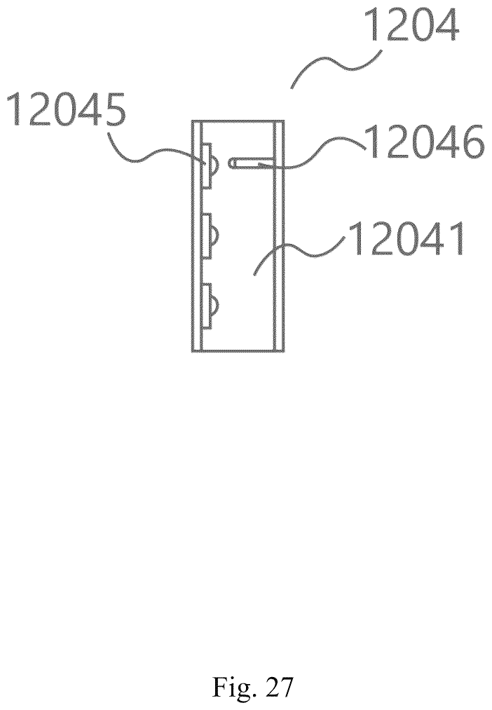

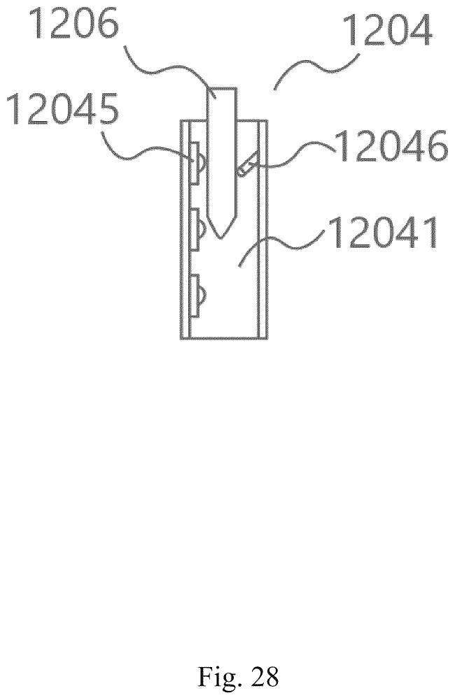

Referring to FIG. 27, this embodiment illustrates a cross-sectional view of an aimer of one embodiment in which the aimer is equipped with displacement detection. The aimer 1204 further includes a fixed rod 12046 and at least one displacement detection roller 12045. One end of the fixed rod 12046 is pivotally connected to an inner wall of the hollow channel 12041, and another end of the fixed rod 12046 is movably disposed in a cavity of the hollow channel 12041. The at least one displacement detection roller 12045 is disposed at the inner wall of the hollow channel 12041, and the at least one displacement detection roller 12045 is located over or beneath the fixed rod 12046. In some embodiments, the hollow channel 12041 in the aimer 1204 is equipped with a fixed rod 12046 and one or more sets of displacement detection rollers 12045. When the instrument passes through the hollow channel, the fixed rod 12046 may be used to hold the instrument against it to achieve a fixed effect, and the displacement detection roller 12045 can calculate a depth of the instrument's passage based on the measurement of its rotation. The displacement detection roller 12045 may be a gear structure, a ball structure, or any other structure that can achieve the same effect. FIG. 28 is a schematic diagram when an instrument 1206 passes through the hollow channel 12041.

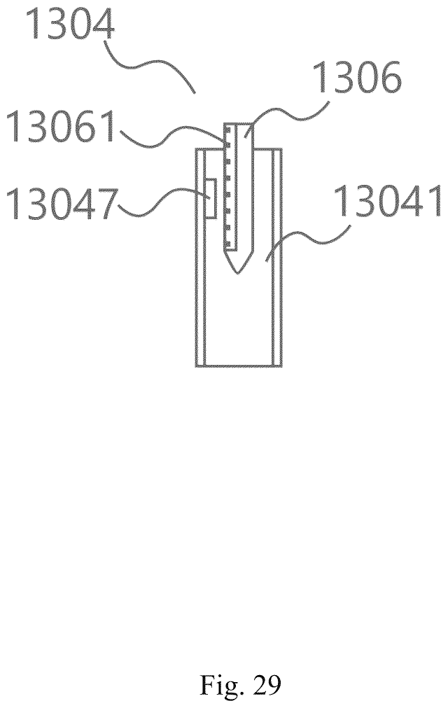

Referring to FIG. 29, this embodiment illustrates a cross-sectional view of an aimer of another embodiment in which the aimer is equipped with displacement detection. The aimer 1304 further includes a scanner 13047, disposed at an inner wall of the hollow channel 13041. The scanner 13047 calculates a depth of the instrument through the hollow channel 13041 by scanning and then calculating by the processor. In some embodiments, the hollow channel 13041 of the aimer 1304 is equipped with the scanner 13047. The scanner may be based on any scanning principle, including but not limited to visible light scanning, infrared light scanning, electromagnetic induction, etc. The surgical instrument 1306 itself has one or more sensors 13061 (or called sensing features) corresponding to the scanner 13047. The sensors 13061 may be scanned and identified by the scanner 13047, and thus the depth of the surgical instrument 1306 passing through the hollow channel 13041 can be acquired through calculation.

Although a series of operations or steps are used to illustrate the method disclosed herein below, the order shown in the operations or steps should not be construed as a limitation of the disclosure. For example, certain operations or steps may be performed in a different order and/or concurrently with other steps. Furthermore, not all illustrated operations, steps, and/or features must be performed to implement embodiments of the present disclosure. Additionally, each operation or step described herein may include several sub-steps or actions.

### Usage Process of Aiming System

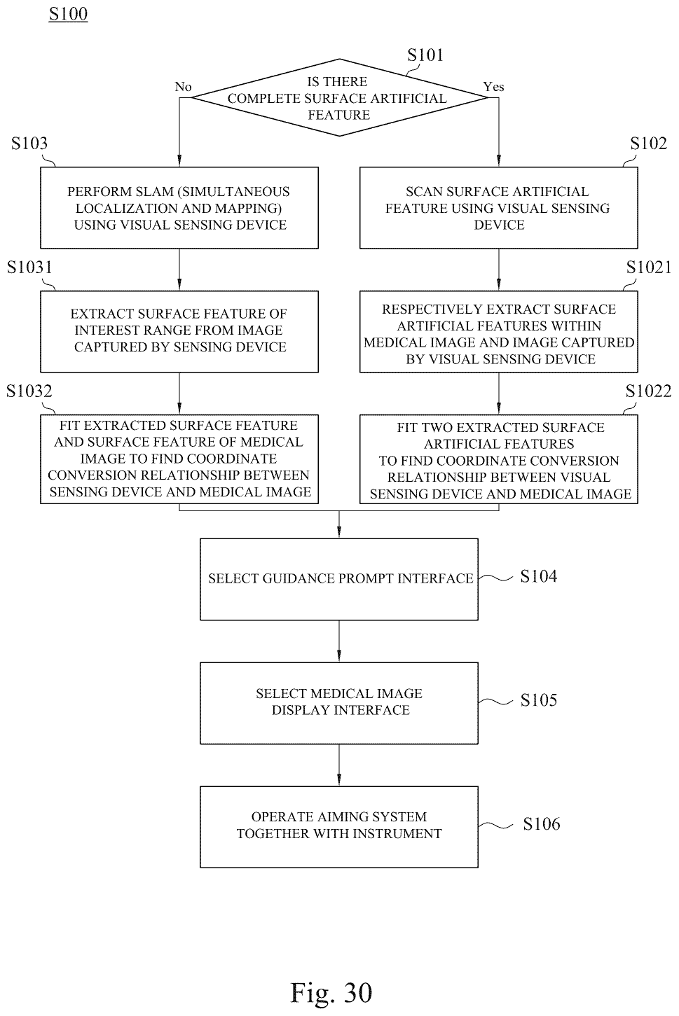

Referring to FIG. 30, a usage process of an aiming system is illustrated. A method S100 of using the aiming system includes providing the aforementioned aiming system; scanning a medical image of an object and determining whether there is a surface artificial feature on the object; if so, scanning the surface artificial feature using the aiming system, and fitting the surface artificial feature on the medical image and the surface artificial feature scanned and acquired by the scanning system to acquire a coordinate conversion relationship; if not, scanning an object surface feature of the object using the aiming system, and fitting the object surface feature on the medical image and the object surface feature scanned and acquired by the scanning system to acquire the coordinate conversion relationship; and operating the aiming system together with an instrument. Specific instructions are as follows.

First, in step S101, it is necessary to determine whether the patient's body surface has an artificial surface feature. The artificial surface feature must have been fixed on the patient's body surface when the patient is scanned for medical imaging of the surgery, and the artificial surface feature must be a material that can be imaged by the medical image, or may be composite materials (i.e., contains an imageable material and a material that can be identified by the positioner, and a relative relationship between the two materials is known). For example, ArUco with a steel ball is used. ArUco is identified by a camera, and the steel ball is identified by computed tomography (CT) imaging. When scanning the medical image of the patient, the artificial surface feature can be captured together. Furthermore, the artificial surface feature may be one or more marking points, and the marking points have high contrast (e.g., black and white areas on the same marking point) and symmetric features (e.g., circles), in which an interval between the black and white areas on each of the marking points may be more than or equal to 1 mm.

If the patient's body surface has the artificial surface feature, step S102 may be entered to scan the surface artificial feature using a visual sensing device. The visual sensing device may be a visible light camera, or further, an RGBD camera.

After the scanning is completed, step S1021 is entered, and the surface artificial feature point on the patient's medical image in step S101 and the surface artificial feature point captured by the visual sensing device in step S102 should be extracted, respectively. After the extraction of the feature points is completed, step S1022 is then entered to fit two groups of the extracted surface artificial feature points to find out a coordinate conversion relationship between the visual sensing device (or the positioner) and the medical image. Specifically, step S1022 includes a real-time positioning step and a registration step, and the order of the two steps is not limited. The real-time positioning step is real-time identification of the artificial feature by the visual sensing device, and the registration step is spatial correspondence between the sensing device and the medical image. The artificial feature includes but is not limited to ArUco, quick response code (QR code), a steel ball or a combination thereof, etc., which can correspond to feature description of spatial coordinates to maintain a rigid spatial relationship with the patient from creation of the medical image to completion of the use of the aimer. In order to enhance robustness of identification of the sensing device, the artificial feature may be a combination of the above types. For example, an ArUco, medical image are printed on a patch including a steel ball, so that the steel ball can be used to identify the feature location, and the visual sensing device can identify the feature location using ArUco.

Following the above step S101, if the patient's body surface does not have an artificial surface feature, step S103 is entered to perform SLAM (simultaneous localization and mapping) scanning using the visual sensing device. After the scanning is completed, step S1031 may be entered to extract a surface feature of an interest range from the captured image.

After the extraction is completed, step S1032 is entered to fit the surface feature (non-artificial surface feature) of the medical image itself in step S101 and the surface feature extracted in step S1031 and then finds out a coordinate conversion relationship between the visual sensing device and the medical image. Specifically, step S1032 includes a real-time positioning step and a registration step, and the order of the two steps is not limited. The real-time positioning step is real-time identification of the patient's body surface feature by a depth sensing device, and the registration step is spatial correspondence between the sensing device and the medical image.

In some implementations, confirmation of the coordinate conversion relationship in step S1022 or step S1032 may be divided into two methods. The first method is to first establish an absolute positioning relationship and then update a relative relationship: the sensing device registers the spatial correspondence between the current feature and the feature at a previous point in time to achieve a subtle convergence error (e.g., convergence to less than or equal to 0.5 mm); the relative relationship is then updated when the sensing device moves subsequently. The second method is to first establish a rough positioning relationship, and then perform a fine positioning relationship: the spatial correspondence between the sensing device and a certain feature on the medical image is roughly positioned (e.g., an error is 3 mm to 5 mm); and fine positioning and convergence error are performed (e.g., convergence to less than or equal to 0.5 mm) when the sensing device moves subsequently. From this, different registration methods may be connected in series, and the latter uses the former as an initial value to achieve effects of optimizing registration results and reducing a number of iterative calculations.

Whether the coordinate conversion relationship between the visual sensing device and the medical image is found through the method in step S1022 or step S1032, after the coordinate conversion relationship is found, step S104 may be entered. In this step, the user can select a guidance prompt interface. More specifically, the guidance prompt interface may include one or more of physical light devices around the hollow channel, displaying through a display, displaying through a projection device, etc. (for details of the guidance prompt interface, please refer to FIGS. 4 to 9 and corresponding descriptions of the embodiments). Furthermore, if the user does not need guidance prompt information, the user can choose not to display the information.

After the guidance prompt interface is determined, step S105 may be entered. In this step, the user can select a medical image display interface according to needs. The medical image display interface may include one or more of displaying by display and displaying by projection, etc. (for a combination relationship between the aiming system and the display or the projection device, please refer to FIGS. 5 to 9 and corresponding descriptions). In the embodiment that displaying by the display, a presentation method on the display may be divided into virtual reality (VR) displaying or mixed reality (MR) displaying. More specifically, VR displaying means that the system can update a digitally reconstructed radiograph (DRR) projection through the calculated coordinate conversion relationship and the current spatial coordinates of the sensing device, and display it on a multiplanar reconstruction (MPR) display; MR displaying refers to superimposing a color image (e.g., a red, green, and blue (RGB) image) acquired through the visual sensing device on the DRR image projected by the VR to achieve the effect of superimposing the two images. Furthermore, if the user does not need the medical imaging information, the user can choose not to display the information.

After solutions of the guidance prompt interface and the medical image display interface are confirmed, step S106 is finally entered. At this time, the user can perform operations using the aiming system together with the instrument.

Furthermore, the advantage of using the visual sensing device with the body surface positioning in the present disclosure is that compared to the use process of the traditional infrared optical or electromagnetic navigation system, the method of the present disclosure does not have the problem of unstable identification due to shielding of the sensed device (e.g., reflective balls, electromagnetic emission devices) and does not interfered by factors of space or environment (e.g., light, magnetic field, etc.).

In addition, most of the traditional infrared optical navigation systems need to fix the fiducial marker on the patient in an invasive manner. However, during the surgery, the positioning may fail due to collision displacement or contamination of the fiducial marker. The method disclosed in the present disclosure does not need to rely on additional invasive fiducial marker to be fixed on the patient, so the risk caused by the failure of the fiducial marker can be avoided.

When the traditional navigation tool is used, it often has to be identified by the tracker, which makes the operation inconvenient. The present disclosure uses the positioner to directly combine with the aimer, which avoids this problem in the operation process and makes surgical guidance more intuitive. Furthermore, the traditional navigation tool needs to design a variety of special trackable markers in order to enable the tool to be navigated. The method disclosed in the present disclosure directly provides the hollow channel for aiming and alignment, so it can be faster and more compatible, and thus can integrate various tools and instruments in the operating room more quickly and with high compatibility.

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. An aiming system, comprising:
a positioner having a function of acquiring spatial information;
an aiming device connected to the positioner; and
a processor connected to the positioner or the aiming device.

2. The aiming system of claim 1, wherein the aiming device comprises an aimer, which comprises at least one hollow channel disposed at a surface of the positioner.

3. The aiming system of claim 2, wherein the positioner comprises at least one sensing device disposed at one side of the positioner.

4. The aiming system of claim 3, wherein an axial direction of the at least one hollow channel and a sight direction of the at least one sensing device are substantially parallel to each other.

5. The aiming system of claim 3, further comprising a prompt signal disposed at one end of the at least one hollow channel, and the prompt signal and the at least one sensing device respectively located at opposite ends of the positioner.

6. The aiming system of claim 5, wherein the prompt signal is annular and has a plurality of light sources, and the prompt signal is arranged around one end of the at least one hollow channel; when a portion of the light sources of the prompt signal emits light, the aiming device moves toward a location where the portion of the light sources are located until the light disappears to complete alignment.

7. The aiming system of claim 3, further comprising a display connected to the aiming device, wherein the display displays image comprising positioning prompt information, medical imaging information, a surgical plan, or a combination thereof.

8. The aiming system of claim 7, wherein the display is disposed on the positioner, and the aimer is penetrated through the display.

9. The aiming system of claim 8, further comprising a prompt signal disposed at one end of the at least one hollow channel, and the prompt signal and the at least one sensing device are respectively located at opposite ends of the positioner.

10. The aiming system of claim 8, wherein the display integrates the aiming device to display a medical image of a body surface range and/or its surroundings corresponding to the aimer at that time, and the medical image comprises a visible light image of the body surface range and/or its surroundings, a fluoroscopic medical image of the body surface range and/or its surroundings, or a combination thereof.

11. The aiming system of claim 7, wherein the display is disposed on the positioner, and the aimer is disposed on the display.

12. The aiming system of claim 1, wherein the aiming device comprises:
a projection light source disposed at a surface of the positioner;
a virtual aimer projected and displayed by the projection light source;
a virtual prompt signal projected and displayed by the projection light source, and the virtual prompt signal presented at one end of the virtual aimer; and
a virtual surgical information projected and displayed by the projection light source, and the virtual surgical information presented at one side of the virtual aimer.

13. The aiming system of claim 1, further comprising at least one handle disposed at one side of the positioner or the aiming device.

14. The aiming system of claim 1, further comprising a connecting rod disposed beneath the positioner or the aiming device, so that the aiming system is locked and fixed in space with any posture.

15. The aiming system of claim 1, further comprising a visible light source disposed at one side of the positioner or one side of the aiming device.

16. The aiming system of claim 1, wherein
the aiming device comprises an aimer, which comprises a hollow channel;
the aiming system further comprises a plurality of slide grooves arranged around outside the hollow channel in sequence; and
the positioner comprises a plurality of sensing devices respectively movably disposed in the plurality of slide grooves and movably arranged around the aimer.

17. The aiming system of claim 16, wherein the aiming device further comprises:
a base, the plurality of slide grooves are arranged around an outer wall of the base in sequence; and
a fine-tuning mechanism disposed between an outer wall of the hollow channel and an inner wall of the base, so that the hollow channel is movably penetrated through the base.

18. The aiming system of claim 1, wherein
the aiming device comprises an aimer, which comprises:
a base; and
a hollow channel movably penetrated through the base; and
the positioner comprises a plurality of sensing devices respectively disposed at an outer surface of the base.

19. The aiming system of claim 18, wherein the base comprises a ball joint, and the hollow channel is rotatably movably penetrated through the ball joint.

20. The aiming system of claim 18, wherein the base comprises a slide rail, and an axial direction of the hollow channel is parallel to an axial direction of the slide rail, and the hollow channel is movably penetrated through the base along the axial direction of the slide rail.

21. The aiming system of claim 1, wherein
the aiming system further comprises a display, which comprises a first side and a second side opposite to the first side;
the aiming device comprises an aimer, which comprises:
a hollow channel disposed on the display or penetrated through the display, and the hollow channel has a first end and a second end opposite to the first end, and the second end of the hollow channel is coplanar with a second end of the display; and
the positioner is disposed on the display.

22. The aiming system of claim 21, wherein the aimer further comprises:
a ball joint disposed at the first end of the hollow channel; and
a suction cup disposed at one side of the ball joint opposite to the first end of the hollow channel.

23. The aiming system of claim 21, wherein the aimer further comprises a sensor disposed at the first end of the hollow channel.

24. The aiming system of claim 21, wherein the positioner protrudes from a surface of the display by a first height, and the hollow channel protrudes from the surface of the display by a second height, and the first height is less than the second height.

25. The aiming system of claim 24, wherein the positioner comprises two sensing devices respectively disposed at both ends of the first side of the display, and there is a distance between the two sensing devices, and the hollow channel has a diameter less than or equal to the distance.

26. The aiming system of claim 21, wherein
the positioner is disposed on the first side of the display; and
the aiming system further comprises an observer disposed on the second side of the display to observe user behavior.

27. The aiming system of claim 21, further comprising a calibration group, which comprises:
a first calibration device, comprising:
a base having a first end and a second end opposite to the first end;
a combination base disposed at the first end of the base, and one end of the display is detachably combined with the combination base; and
a bump protruding from the second end of the base, and the bump having a calibration feature located in front of the first end of the hollow channel,
wherein the positioner is movably disposed on the first side of the display to perform calibration according to a position of the calibration feature;
a second calibration device, comprising:
a rod body having a first end and a second end opposite to the first end, the second end detachably penetrated through and in the hollow channel or sleeved outside the hollow channel; and
a calibration feature disposed at the first end of the rod body, the calibration feature located in front of the first end of the hollow channel,
wherein the hollow channel moves or rotates along an axial direction to perform calibration according to a position of the calibration feature; or
a third calibration device, comprising:
a base having a first end and a second end opposite to the first end;
a combination base disposed at the first end of the base, one end of the display detachably combined with the combination base;
a rod body having a first end and a second end opposite to the first end, the second end of the rod body sliding or rotating along an axial direction of the base; and
a calibration feature disposed at the first end of the rod body, the calibration feature located in front of the first end of the hollow channel,
wherein the positioner is fixed, and the calibration feature on the rod body can slide and rotate to perform calibration.

28. The aiming system of claim 1, wherein
the positioner comprises a first side, a second side opposite to the first side, and at least one camera disposed at the first side;
the aiming device comprises an aimer, which comprises:
a hollow channel disposed on the positioner, and the hollow channel has a first end and a second end opposite to the first end, and the second end is coplanar with a second side of the positioner, and the first end of the hollow channel protrudes from the first side of the positioner; and
a calibration feature disposed at the first end of the hollow channel, and a distance between the calibration feature and the first side of the positioner is greater than or equal to a shortest focal length of the at least one camera.

29. The aiming system of claim 1, wherein the aiming device comprises an aimer, which comprises a hollow channel disposed at one side of the positioner, wherein an inner diameter of the hollow channel is greater than or equal to an outer diameter of an instrument, wherein the aimer further comprises:
an inflatable structure disposed at an inner wall of the hollow channel;
at least one vacuum feature disposed at the inner wall of the hollow channel, and when the at least one vacuum feature is in a vacuum state, the at least one vacuum feature presents a hardened state, and when the at least one vacuum feature is in a non-vacuum state, the at least one vacuum feature presents a soft state;
an adapter, the adapter being annular and disposed at the inner wall of the hollow channel; an inner diameter of the adapter being greater than or equal to the outer diameter of the instrument;
the adapter and a dedicated drape, the adapter being annular and disposed at the inner wall of the hollow channel; the inner diameter of the adapter being greater than or equal to the outer diameter of the instrument; the dedicated drape being disposed between the hollow channel and the adapter;
a fixed rod and at least one displacement detection roller, one end of the fixed rod being pivotally connected to the inner wall of the hollow channel, another end of the fixed rod being movably disposed in a cavity of the hollow channel; the at least one displacement detection roller being disposed at the inner wall of the hollow channel, and the at least one displacement detection roller being not located over or beneath the fixed rod; or
a scanner disposed at the inner wall of the hollow channel, the scanner acquiring a depth of the instrument through the hollow channel by scanning and then calculating by the processor.

30. A method of using an aiming system, comprising:
providing the aiming system of claim 1;
scanning a medical image of an object and determining whether there is a surface artificial feature on the object,
if so, scanning the surface artificial feature using the aiming system, and fitting the surface artificial feature on the medical image and the surface artificial feature scanned and acquired by the scanning system to acquire a coordinate conversion relationship;
if not, scanning an object surface feature of the object using the aiming system, and fitting the object surface feature on the medical image and the object surface feature scanned and acquired by the scanning system to acquire the coordinate conversion relationship; and operating the aiming system together with an instrument.

31. The method of claim 30, wherein after the step of determining whether there is the surface artificial feature on the object, the method further comprises:
selecting a guidance prompt interface, which comprises displaying through a prompt signal of the aiming system, a display displaying through the aiming system, a projection light source projection displaying through the aiming device of the aiming system, or a combination of one or more thereof.

32. The method of claim 30, wherein after the step of determining whether there is the surface artificial feature on the object, the method further comprises:
selecting a medical image display interface, which comprises displaying by a display of the aiming system, displaying by a projection light source projection of the aiming device of the aiming system, or a combination of one or more thereof.

33. The method of claim 32, wherein the display displaying comprises a virtual reality displaying or a mixed reality displaying,
wherein the virtual reality displaying is a digitally reconstructed radiograph updated by the aiming system through the coordinate conversion relationship and a spatial coordinate of the positioner of the aiming system at that time and displayed on the display,
wherein the mixed reality displaying is acquired by superimposing a color image acquired through the positioner of the aiming system on the digitally reconstructed radiograph projected by the virtual reality displaying, so that the digitally reconstructed radiograph and the color image are superimposed.
